(19) United States
(12) Patent Application Publication
(10) Pub. No.: US 2022/0236291 A1
(43) Pub. Date: Jul. 28, 2022

(54) DETECTION OF NEURAL-DERIVED DEBRIS IN RECIRCULATING PHAGOCYTES

(71) Applicant: ZELOSDX, INC., Tucson, AZ (US)

(72) Inventors: Uwe R. Muller, Gallatin, TN (US); Vanessa White, Tucson, AZ (US)

(21) Appl. No.: 17/719,177

(22) Filed: Apr. 12, 2022

Related U.S. Application Data
(63) Continuation-in-part of application No. 17/228,416, filed on Apr. 12, 2021.
Application No. 17/228,416 is a continuation-in-part of application No. 16/271,186, filed on Feb. 8, 2019, now abandoned.
Application No. 16/271,186 is a continuation-in-part of application No. 16/872,064, filed on May 11, 2020, now abandoned.
Application No. 16/872,064 is a continuation-in-part of application No. 16/271,186, filed on Feb. 8, 2019, now abandoned.
Application No. 16/271,186 is a continuation-in-part of application No. 15/472,066, filed on Mar. 28, 2017, now abandoned.
Application No. 15/472,066 is a continuation-in-part of application No. 14/721,250, filed on May 26, 2015, now abandoned.
Application No. 14/721,250 is a continuation-in-part of application No. 14/704,791, filed on May 5, 2015, now abandoned.
Application No. 14/704,791 is a continuation-in-part of application No. PCT/US13/68465, filed on Nov. 5, 2013.
Application No. PCT/US13/68465 is a continuation-in-part of application No. 13/852,889, filed on Mar. 28, 2013, now abandoned.
Application No. 13/852,889 is a continuation-in-part of application No. 12/325,035, filed on Nov. 28, 2008, now Pat. No. 8,506,933.
Application No. 14/721,250 is a continuation-in-part of application No. 13/645,266, filed on Oct. 4, 2012, now abandoned.
Application No. 13/645,266 is a continuation-in-part of application No. 12/853,203, filed on Aug. 9, 2010, now abandoned.
Application No. 13/645,266 is a continuation-in-part of application No. 12/954,396, filed on Nov. 24, 2010, now abandoned.
Application No. 14/721,250 is a continuation-in-part of application No. 12/954,505, filed on Nov. 24, 2010, now abandoned.
Application No. 14/704,791 is a continuation-in-part of application No. 12/954,505, filed on Nov. 24, 2010, now abandoned.
(60) Provisional application No. 62/845,670, filed on May 9, 2019.
(60) Provisional application No. 61/722,441, filed on Nov. 5, 2012.
(60) Provisional application No. 62/086,948, filed on Dec. 3, 2014.
(60) Provisional application No. 61/650,947, filed on May 23, 2012.
(60) Provisional application No. 60/991,594, filed on Nov. 30, 2007.
(60) Provisional application No. 61/007,728, filed on Dec. 14, 2007.
(60) Provisional application No. 61/020,820, filed on Jan. 14, 2008.
(60) Provisional application No. 61/042,407, filed on Apr. 4, 2008.
(60) Provisional application No. 61/232,605, filed on Aug. 10, 2009.
(60) Provisional application No. 61/264,763, filed on Nov. 27, 2009.
(60) Provisional application No. 61/264,760, filed on Nov. 27, 2009.
(60) Provisional application No. 61/371,122, filed on Aug. 5, 2010.
(60) Provisional application No. 61/393,254, filed on Oct. 14, 2010.

(51) Int. Cl. G01N 33/68; G01N 33/569; G01N 33/50

(52) U.S. Cl. CPC G01N 33/6896; G01N 2800/2835; G01N 33/5055; G01N 33/56972

(57) ABSTRACT

Methods for preparing neural-derived compounds, e.g., the debris such as peptides, nucleic acids, or other compounds that would only normally be found in brain or CNS tissue, from circulating phagocytes. The methods herein may feature extracting lysate from circulating phagocytes obtained from outside central nervous system (CNS) tissue, producing a fraction of the lysate comprising CNS-derived compounds, and analyzing the CNS-derived compounds in the fraction.

### CROSS REFERENCE

This application is a continuation-in-part and claims benefit of U.S. patent application Ser. No. 17/228,416 filed on Apr. 12, 2021, which is a continuation-in-part and claims benefit of U.S. patent application Ser. No. 16/271,186 filed on Feb. 8, 2019, the specification(s) of which is/are incorporated herein in their entirety by reference.

This application is also a continuation-in-part and claims benefit of U.S. patent application Ser. No. 16/872,064 filed on May 11, 2020, which is a non-provisional and claims benefit of U.S. Provisional Patent Application No. 62/845,670, filed May 9, 2019, the specification(s) of which is/are incorporated herein in their entirety by reference. The application Ser. No. 16/872,064 is also a continuation-in-part and claims benefit of U.S. patent application Ser. No. 16/271,186 filed on Feb. 8, 2019, the specification(s) of which is/are incorporated herein in their entirety by reference.

The application Ser. No. 16/271,186 is a continuation-in-part and claims benefit of U.S. patent application Ser. No. 15/472,066 filed on Mar. 28, 2017, which is a continuation-in-part and claims benefit of U.S. patent application Ser. No. 14/721,250 filed on May 26, 2015, which is a continuation-in-part and claims benefit of U.S. patent application Ser. No.14/704,791 filed on May 5, 2015, which is a continuation-in-part of PCT Application No. PCT/US13/68465 filed on Nov. 5, 2013, which claims priority to U.S. Provisional Patent Application No. 61/722,441 filed on Nov. 5, 2012, the specification(s) of which is/are incorporated herein in their entirety by reference.

The application Ser. No. 14/721,250 is also a non-provisional and claims benefit of U.S. Provisional Patent Application No. 62/086,948 filed Dec. 3, 2014, the specification of which is incorporated herein in its entirety by reference.

The application Ser. No. 14/721,250 is also a continuation-in-part and claims benefit of U.S. patent application Ser. No. 13/852,889 filed on Mar. 28, 2013, which is a non-provisional and claims benefit of U.S. Provisional Patent Application No. 61/650,947 filed May 23, 2012, the specification of which is incorporated herein in its entirety by reference. The application Ser. No. 13/852,889 is also a continuation-in-part and claims benefit of U.S. Patent Application No. 12/325,035 filed on Nov. 28, 2008, now U.S. Pat. No. 8,506,933, which is a non-provisional and claims benefit of U.S. Provisional Patent Application No. 60/991,594 filed Nov. 30, 2007, U.S. Provisional Patent Application No. 61/007,728 filed Dec. 14, 2007, U.S. Provisional Patent Application No. 61/020,820 filed Jan. 14, 2008, and U.S. Provisional Patent Application No. 61/042,407 filed on Apr. 4, 2008, the specification(s) of which is/are incorporated herein in their entirety by reference.

The application Ser. No. 14/721,250 is also a continuation-in-part and claims benefit of U.S. patent application Ser. No. 13/645,266 filed on Oct. 4, 2012, which is a continuation-in-part and claims benefit of U.S. patent application Ser. No. 12/853,203 filed on Aug. 9, 2010, which is a non-provisional and claims benefit of U.S. Provisional Patent Application No. 61/232,605 filed Aug. 10, 2009, the specification(s) of which is/are incorporated herein in their entirety by reference. The application Ser. No. 13/645,266 is also a continuation-in-part and claims benefit of U.S. patent application Ser. No. 12/954,396 filed on Nov. 24, 2010, which is a non-provisional and claims benefit of U.S. Provisional Patent Application No. 61/264,763 filed Nov. 27, 2009, the specification(s) of which is/are incorporated herein in their entirety by reference.

The application Ser. No. 14/721,250 is also a continuation-in-part and claims benefit of U.S. patent application Ser. No. 12/954,505 filed on Nov. 24, 2010, the specification(s) of which is/are incorporated herein in their entirety by reference. The application Ser. No. 14/704,791 is also a continuation-in-part and claims benefit of U.S. patent application Ser. No. 12/954,505 filed on Nov. 24, 2010, the specification(s) of which is/are incorporated herein in their entirety by reference. The application Ser. No. 12/954,505 is a non-provisional and claims benefit of U.S. Provisional Patent Application No. 61/264,760 filed Nov. 27, 2009, U.S. Provisional Patent Application No. 61/371,122 filed Aug. 5, 2010, and U.S. Provisional Patent Application No. 61/393,254 filed on Oct. 14, 2010, the specification(s) of which is/are incorporated herein in their entirety by reference.

### BACKGROUND OF THE INVENTION

### Field of the Invention

The present invention relates to preparation of compounds (e.g., proteins and/or other molecules) derived from neural tissue, wherein the compounds are inside or displayed on the cell surface of recirculating phagocytes. The present invention may include whole sample analysis, single-cell analysis, etc.

### Background Art

In general, when tissue damage occurs, it incites inflammation, which usually aids in wound healing. For example, one of the normal functions of inflammation is to recruit phagocytes to clear away the cellular debris and prepare the injured site for repair and rebuilding. These phagocytes may be resident in the brain (e.g., dendritic cells, microglial cells) or recruited from the bloodstream (e.g., monocytes). Cells that engulf debris are thought to enter the brain by crossing the blood-brain barrier but were previously not believed to return to the bloodstream. Inventors previously discovered that said debris-laden phagocytes may re-enter the bloodstream from the brain, and it is possible to detect, measure, monitor, and/or analyze said brain-derived or CNS-derived debris from the phagocytic cells.

The debris can be indicative of processes occurring in the central nervous system (CNS) (e.g., brain tissue). For example, the presence and/or amount of the debris may be associated with various states of the brain, e.g., biological changes in the brain related to active central nervous system tissue damage, active central nervous system repair, active neurodegeneration, normal CNS processes, aging, etc. (in apparently healthy and/or diseased samples).

Thus, the presence and/or amount of the debris may be used for monitoring biological changes in the brain, such as biological changes associated with normal aging, neurological trauma, or neurological disease (e.g., neurodegenerative diseases, CNS tissue damage, CNS tissue repair, etc.). For example, the present invention may be used to monitor aging processes in the brain. The present invention may be used to monitor worsening or improvement of a particular brain condition or neurological disease, or response to a treatment. The presence and/or amount of the debris may be compared to a threshold to determine an amount of change relative to a baseline or threshold. For example, the change may be based on a subject's (e.g., patient's, animal's) baseline levels of the debris as detected at a previous time, a change from time T1 to time T2, an industry standard, etc. For example, each patient may have his/her own baseline levels (e.g., levels of the biomarker or panel of biomarkers, % of cells positive for the biomarker or panel of biomarkers, etc.). The levels (relative to baseline, for example) may increase, which in some embodiments may be related to a biological change in the CNS tissue (e.g., aging, disease, etc.). The levels may decrease, which in some embodiments may be related to a positive effect of a treatment. In some embodiments, the methods herein are utilized for cross-sectional studies wherein comparisons are made at a single point in time. In some embodiments, the methods herein are utilized for longitudinal studies wherein comparisons are made over time.

The present invention is not limited to humans. As used herein, a patient or subject may refer to an animal such as but not limited to a mammal. Mammals may include but are not limited to primates (e.g., a human, non-human primates), a mouse, a rat, a llama, a rabbit, a dog, a primate, a guinea pig, a cat, a hamster, a pig, a goat, a horse, or a cow. The present invention is not limited to the aforementioned subjects or patients.

### SUMMARY OF THE INVENTION

The present invention describes a phagocytic shuttle method (PSM) wherein the phagocytes that re-enter the bloodstream from the central nervous system (CNS) tissues (e.g., brain tissue) are shuttles for CNS-derived (e.g., brain-derived, neural-derived) debris. The methods herein describe preparation of the CNS-derived debris for analysis. Non-limiting examples of methods described herein include flow cytometry, ELISA, FACS, and fluorescent staining. In some embodiments, the analysis is single cell analysis, e.g., using flow cytometry, single cell ELISA, etc. Such techniques are known in the art. For example, in single cell ELISA, cells are captured and immobilized. In some cases, the cells are permeabilized and antibodies are added directed to biomarkers and subsequently subjected to imaging (e.g., via microscopy, direct imaging, etc.). In some cases, the cells are immobilized and lysed, and biomarkers are captured via specific antibodies that have also been immobilized on the same surface. After washing to remove unbound materials, a secondary labeled antibody specific for the biomarker(s) is added, which after washing is then analyzed by imaging. The present invention includes the use of systems (e.g., microfluidic devices, etc.) that allow single cell trapping and analysis by ELISA. Details can be found in Yin and Marshall (2012, Cur. Op. Biotechnology 23:110-119), Spiller et al. (2010, Nature 465:736-745), among others. The present invention includes the use of systems (e.g., microfluidic devices, etc.) that allow single cell trapping and analysis by ELISA. The present invention is not limited to these particular methods or systems for single cell analysis.

Without wishing to limit the present invention to any theory or mechanism, the present invention may provide close to real-time data on what is happening in the brain since that particular cargo may only be present in the recirculating phagocytes for a certain length of time before it is digested (e.g., partially digested or fragmented, completely digested, etc.).

The present invention features preparation of phagocytes containing CNS-derived (e.g., brain-derived, neural-derived) compounds (e.g., the debris or biomarkers that would only normally be found in CNS tissue such as but not limited to brain) for analysis. The present invention also features preparation of the CNS-derived compounds found in the circulating phagocytes. The present invention also features preparation of blood samples for analyzing the CNS-derived compounds found in the circulating phagocytes. The present invention is not limited to isolation of circulating phagocytes and creating a lysate. The present invention also includes methods using whole blood. The present invention also includes methods for single-cell analysis.

The methods herein for preparing central nervous system (CNS)-derived (e.g., brain-derived) compounds may comprise introducing to a whole blood sample obtained from outside central nervous system (CNS) tissue of a subject a first detectable binding moiety specific for circulating phagocytes and a second detectable binding moiety specific for a CNS-derived molecule, the first detectable binding moiety being differentially detectable from the second detectable binding moiety; subjecting the preparation to single-cell analysis for detecting the first detectable binding moiety and second detectable binding moiety; and analyzing the CNS-derived molecules in the preparation.

In some embodiments, the single-cell analysis is flow cytometry. In some embodiments, the single cell analysis is single cell ELISA. In some embodiments, the single-cell analysis is based on microscopy. In some embodiments, the single-cell analysis comprises placing the preparation on a solid surface, using said surface as a wave guide for illumination, and imaging by direct charge-coupled device (CCD). In some embodiments, the CNS-derived compounds are peptides, whole proteins, epitopes of a protein or peptide, lipids, membrane components, nucleic acids, metabolites, toxins, infectious agents, or a combination thereof. In some embodiments, the circulating phagocytes are macrophages, monocytes or a subgroup thereof, dendritic cells, neutrophils, or a combination thereof. In some embodiments, the first detectable binding moiety, the second detectable binding moiety, or both comprise a fluorescent label, a fluorescent antibody, a nanoparticle, a quantum dot, or a tag. In some embodiments, the CNS-derived molecule is GFAP. In some embodiments, the CNS-derived molecule is Tau. In some embodiments, the CNS-derived molecule is GFAP, Tau, or both. In some embodiments, the CNS-derived molecule comprises one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin All, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.

In some embodiments, the method comprises producing a preparation comprising CNS-derived molecules by introducing to a whole blood sample obtained from outside central nervous system (CNS) tissue of a subject a first detectable binding moiety specific for circulating phagocytes and a second detectable binding moiety specific for a CNS-derived molecule, the first detectable binding moiety being differentially detectable from the second detectable binding moiety; and analyzing the CNS-derived molecules in the preparation.

The methods herein for preparing and/or analyzing central nervous system (CNS)-derived (e.g., brain-derived) compounds may comprise single-cell analysis of circulating phagocytes from a fluid sample obtained from outside central nervous system (CNS) tissue of a subject; and analysis of the CNS-derived compounds in the cells.

The present invention also provides methods for preparing and/or analyzing CNS-derived compounds wherein the CNS-derived compound is displayed on the cell surface of the phagocytes. For example, the method may comprise extracting circulating phagocytes from a fluid sample obtained from outside central nervous system (CNS) tissue of a subject; and producing a fraction of the extracted circulating phagocytes by separating phagocytes with membrane-bound CNS-derived peptides/compounds from phagocytes without membrane-bound CNS-derived peptides/compounds. The fraction of the phagocytes may comprise the phagocytes with membrane-bound CNS-derived peptides/compounds. In some embodiments, the method further comprises analyzing the phagocytes in the fraction. In some embodiments, the method comprises lysing the whole sample as described herein, rather than first extracting the circulating phagocytes. In some embodiments, the method comprises single-cell analysis as described herein.

In some embodiments, a sample for the methods of the present invention is prepared using a filtration system, e.g., a sample fraction or blood fraction is produced using a filtration system. In some embodiments, a sample is prepared using a magnetic bead system, e.g., a sample fraction or blood fraction is produced using a magnetic bead system. In some embodiments, a sample is prepared using a chromatography system, e.g., a sample fraction or blood fraction is produced using a chromatography system. In some embodiments, a sample is prepared using a nanoparticle system, e.g., a sample fraction or blood fraction is produced using a nanoparticle system.

In some embodiments, the circulating phagocytes are macrophages. In some embodiments, the circulating phagocytes are dendritic cells. In some embodiments, the circulating phagocytes are monocytes (or subgroups thereof, e.g., CD16+ monocytes). In some embodiments, the circulating phagocytes are granulocytes, e.g., neutrophils. In some embodiments, the phagocytes are a combination of cells, such as macrophages, monocytes, and neutrophils. In some embodiments, the phagocytes comprise a combination of cells, such as cells in PBMC preparations and neutrophils. In some embodiments, the circulating phagocytes are macrophages, monocytes (or subgroups thereof), neutrophils, dendritic cells, or a combination thereof.

In some embodiments, the circulating phagocytes are obtained and/or isolated using an affinity chromatography system. For example, the affinity chromatography system may comprise a phagocyte-specific antibody bound to a slide. In some embodiments, the affinity chromatography system comprises a phagocyte-specific antibody bound to a resin in a column. In some embodiments, the circulating phagocytes are obtained using a spin column. In some embodiments, the circulating phagocytes are obtained using a magnetic bead system. In some embodiments, the circulating phagocytes are obtained using a nanoparticle system. In some embodiments, the circulating phagocytes are obtained using forward-scattered light or side-scattered light in flow cytometry. In some embodiments, the circulating phagocytes are obtained using a fluorescence system.

For any of the embodiments herein, the CNS-derived compound or antigen may be one or more of the following compounds: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, a tyrosinase, tyrosinase EC 1.14.18.1, tyrosine hydroxylase, tyrosinase EC 1.14.16.2 (tyrosine 3-monooxygenase etc.), a synaptic antigen (e.g., PSD-95 protein, neurogranin, SNAP-25, TDP-43, etc.), transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin All, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), a breakdown product thereof, a fragment or fragments thereof, the like, biomarkers associated with neurological diseases that will be identified in the future, a combination thereof, etc. The present invention is not limited to the aforementioned biomarkers or antigens. The biomarker may be selected based on its association with a particular disease or condition.

The methods herein for preparing central nervous system (CNS)-derived (e.g., brain-derived) compounds may comprise lysing whole blood and analyzing the CNS-derived compounds in the lysate or a fraction thereof. In some embodiments, the methods comprise extracting lysate from circulating phagocytes from a fluid sample obtained from outside central nervous system (CNS) tissue of a subject; and producing a fraction of the lysate by selectively collecting CNS-derived (e.g., brain-derived) compounds, wherein the fraction comprises CNS-derived (e.g., brain-derived) compounds. In some embodiments, the method further comprises analyzing the CNS-derived compounds in the fraction.

The present invention also features methods for preservation of samples for preserving the amount and/or structure and/or location of the CNS-derived biomarker(s) of interest (e.g., for preserving the amount and/or structure and/or location of the epitope(s) of interest). For example, the present invention provides methods for treating samples for the purposes of preserving the biomarker, e.g., via heat denaturation (wherein proteolytic enzymes or other factors are inhibited without affecting the biomarker, e.g., the epitope of the biomarker, to a large extent). Other methods of preservation may include freeze drying or other rapid freezing processes, application of heparin or other factors, modifying the pH of the sample, etc. The present invention is not limited to the aforementioned methods or compositions.

The term “predetermined threshold,” as used herein, may refer to an industry standard, a laboratory standard, a patient standard (e.g., the predetermined threshold is a level of the biomarker in phagocytes isolated from a fluid sample obtained from the patient before administration of the therapeutic compositions or before a second time point, etc.), or other appropriate standard. In some embodiments, the level of the biomarker is compared to a predetermined threshold to determine if it is normal, abnormal, changed, unchanged (e.g., relative to a previous result), etc. In certain embodiments, a predetermined threshold is a patient's result from a previous time point, and the sample of interest is compared to said previous result. As previously discussed, the patient may refer to a human patient or an animal.

Without wishing to limit the present invention to any theory or mechanism, it is believed that biomarkers that are associated with particular disease states of interest (e.g., biomarkers found in the re-circulating phagocytes as described herein) will continue to be discovered. Since the methods herein are not necessarily limited by the particular biomarker but instead features the phagocytic shuttle method (e.g., wherein the phagocytes are shuttles for CNS-derived debris indicative of processes occurring in the CNS) and steps for isolating the biomarkers within the shuttle phagocytes, the present invention includes those biomarkers that will be discovered in the future. The present invention also includes panels of biomarkers, e.g., combinations of biomarkers relevant for the analysis. The panel of biomarkers may comprise two or more biomarkers, three or more, four or more, five or more, six or more, seven or more, eight or more, nine or more, 10 or more, 15 or more, 20 or more, 30 or more 40 or more, 50 or more biomarkers, etc.

The present invention also features the use of nanoparticles. Nanoparticles may be used to determine the presence and/or amount of a particular biomarker (e.g., epitope) in a particular cell or group of cells. In some embodiments, the nanoparticles are noble metal nanoparticles or alloys of noble metals. In some embodiments, the nanoparticles are gold nanoparticles, silver nanoparticles, or a combination thereof. In some embodiments, the nanoparticles are rods, spheres, or a combination thereof. In some embodiments, the nanoparticles have a diameter of 2 nm to 250 nm. In some embodiments, the biophysical properties refer to the adsorption or emission of electromagnetic waves by the nanoparticles in response to incident electromagnetic waves. In some embodiments, the biophysical properties refer to surface plasmon resonance. In some embodiments, the differential biophysical properties are measured by dynamic light scattering or tunable resistive pulse sensing.

For example, the present invention also features a method comprising extracting lysate from circulating phagocytes from a fluid sample obtained from outside central nervous system (CNS) tissue of a subject; adding a first nanoparticle that is coated with an antibody to a specific single epitope on the biomarker molecule; and adding a second nanoparticle coated with an antibody specific to a different specific single epitope on the biomarker molecule. In some embodiments, the binding of both types of nanoparticles to the same biomarker molecule result in both nanoparticles being in close proximity such that the biophysical properties of the nanoparticle-biomarker complex changes detectably from the biophysical properties of the unbound nanoparticles.

The present invention also features a method comprising lysing all cells in a fluid sample (e.g., whole blood) obtained from outside central nervous system (CNS) tissue of a subject; isolating membrane fragments of lysed circulating phagocytes in said fluid sample by capture via their specific cell surface markers; and analyzing said membrane fragments for membrane-bound CNS-derived peptides or compounds.

The present invention also features a method comprising treating a fluid sample obtained from outside central nervous system (CNS) tissue of a subject with a mixture of antibodies specific for phagocyte cell surface markers and brain derived biomarkers, whereby the cell surface marker specific antibodies are labeled with a label moiety A and the antibodies specific for brain derived biomarkers are labeled with a different label moiety B; and determining the moiety ratio of phagocytes or cell fragments of phagocytes with both label moieties (A and B) to phagocytes or phagocyte cell fragments with only the cell surface specific moiety (A). In some embodiments, the fluid sample is treated with a fixative after addition of cell surface marker specific antibodies and before addition of the biomarker specific antibodies. In some embodiments, the fluid sample is further treated with a cell permeabilization reagent before addition of the biomarker specific antibodies. In some embodiments, the fluid sample is treated with a cell lysing agent post antibody treatment In some embodiments, the fluid sample is treated with a lysing agent prior to addition of antibodies. In some embodiments, the label moieties are fluorescent moieties. In some embodiments, the label moieties are nanoparticles. In some embodiments, the label nanoparticles are detected by their spectral response to excitation by an electromagnetic wave. In some embodiments, the label moieties are quantum dots. In some embodiments, the labels are colorimetric moieties.

The present invention is not limited to fluorescent assays, e.g., fluorescent microscopy or imaging. In some embodiments, the methods herein comprise colorimetric assays. As a non-limiting example, the methods may comprise a colorimetric ELISA. In some embodiments, the methods herein comprise imaging without a microscope. In some embodiments, the methods herein comprise using an image analysis system, which may provide images from surfaces such as a slide or a plate (e.g., microplate well), etc.

The present invention also features the use of a biomarker isolated from circulating phagocytes collected from a fluid sample derived from a subject having or suspected of having biological changes in the brain or other CNS tissue, such as biological changes associated with central nervous system tissue damage, central nervous system repair, neurodegeneration, aging, normal processes, etc., as described herein, wherein the fluid sample is from outside of a brain tissue of the subject. The biomarker may be used in a method of confirming presence of central nervous system damage or central nervous system death. The biomarker may be used in a method of characterizing a state of one or more nerves in the brain tissue (e.g., nerve death).

The present invention also features methods of validating a correlation between a biomarker and a biological change in the brain or CNS tissue, such as one associated with central nervous system tissue damage, central nervous system repair, neurodegeneration, aging, or other CNS processes. In some embodiments, the method comprises analyzing levels of the CNS-derived biomarker from circulating phagocytes, e.g., using any of the methods described herein. In some embodiments, an abnormal level of the biomarker relative to a control may validate the correlation between the biomarker and the CNS process. Non-limiting examples of CNS processes include TBI, CTE, Parkinson's disease, mild cognitive impairment, normal aging brain, Alzheimer's disease, PTSD, sleep deprivation, glioblastoma, a process related to an implantable device, neurostimulation, normal activity, etc.

The present invention also includes the use of a CNS-derived biomarker isolated from or analyzed from circulating phagocytes collected from a fluid sample derived from a subject. The subject may be suspected of having experienced a biological change in the brain or CNS tissue, such as one associated with central nervous system tissue damage, central nervous system repair, neurodegeneration, or aging. The subject may be neurologically healthy.

The methods herein may be used for methods of detecting biological changes in CNS tissue. The methods may be performed in lieu of obtaining imaging of the subject or obtaining a biopsy.

In some embodiments, the biological changes in the CNS tissue are associated with aging or normal activity. In some embodiments, the biological changes in the CNS tissue are associated with tissue damage, neurological disease, trauma. In some embodiments, the biological changes in the CNS tissue are associated with neurodegeneration, Multiple Sclerosis, Alzheimer's disease, mild cognitive impairment, Parkinson's disease, Multiple System Atrophy, Lewy body Disease, Progressive Supranuclear Atrophy, Corticobasal Degeneration, Amyotrophic Lateral Sclerosis, Huntington's Disease, concussion, Traumatic Brain Injury, REM sleep behavior disorder, cancer (e.g., primary or secondary), or a disease causing secondary central nervous system damage. The biological changes may be associated with cognitive impairment, motor disturbances, or both.

The present invention also provides a method comprising producing a preparation comprising CNS-derived molecules by introducing to a sample obtained from outside central nervous system (CNS) tissue of a subject a first detectable binding moiety specific for circulating phagocytes and a second detectable binding moiety specific for a CNS-derived molecule, the first detectable binding moiety being differentially detectable from the second detectable binding moiety; and analyzing the CNS-derived molecules in the preparation.

In some embodiments, the CNS-derived compound is an epitope of a protein or peptide (or an epitope of a breakdown product of a protein or peptide). In some embodiments, the CNS-derived compound is a peptide, whole protein, lipid, membrane component, nucleic acid, metabolite, toxin, infectious agent, or a combination thereof. In some embodiments, the sample is isolated circulating phagocytes. In some embodiments, the sample is lysed circulating phagocytes. In some embodiments, the sample is whole blood. In some embodiments, the sample is a portion of blood. In some embodiments, the sample is lysed whole blood or lysed blood portion. In some embodiments, the circulating phagocytes are macrophages, monocytes or a subgroup thereof, dendritic cells, neutrophils, or a combination thereof. In some embodiments, the sample is fixed to a surface, e.g., a slide, plate, filter, a resin, the like, etc. In some embodiments, the detectable binding moiety specific for circulating phagocytes is bound to a solid support, e.g., a slide, plate, filter, a resin, the like, etc. In some embodiments, the first detectable binding moiety, the second detectable binding moiety, or both comprise a fluorescent label or fluorescent antibody. In some embodiments, the first detectable binding moiety, the second detectable binding moiety, or both comprise a nanoparticle or quantum dot. In some embodiments, the first detectable binding moiety, the second detectable binding moiety, or both comprise a tag.

In some embodiments, analyzing the CNS-derived molecules in the preparation of cells comprises measuring light frequencies of the preparation of cells to detect proximity of the nanoparticles. In some embodiments, the sample is subjected to affinity chromatography. In some embodiments, the sample is subjected to a magnetic bead system.

In some embodiments, analyzing the CNS-derived molecules in the preparation of cells comprises ELISA. In some embodiments, analyzing the CNS-derived molecules in the preparation of cells comprises microscopy, e.g., fluorescence microscopy, colorimetric microscopy, etc. In some embodiments, analyzing the CNS-derived molecules in the preparation of cells comprises flow cytometry, e.g., fluorescence activated cell sorting (FACS).

In some embodiments, the CNS-derived molecule is amyloid beta. In some embodiments, the CNS-derived molecule is GFAP.

Embodiments of the present invention can be freely combined with each other if they are not mutually exclusive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the distribution of PBMC Tau levels. Normalized signal intensities of multiple assays for each sample were averaged. The average for each group is shown with a bar indicating the standard deviation. The average normalized buffer control of 42 independent assays is also shown to demonstrate the significance of the assay results.

FIG. 2 shows a comparison of GFAP concentrations in rats before and after implantation of microelectrodes. PBMCs were isolated from peripheral blood of rats before and after implantation of the 4 microelectrodes into the brains of 2 male rats in a square 1 mm apart. Electro-stimulation began 48 hours later (1 hr each day, 4 weeks total). The level of GFAP was determined in 2 female rats (F) and the two male rats (M) by ELISA before and at the indicated times after electrode implantation.

FIG. 3 shows western blot analysis of PBMC Extracts. Protein standards and extracts were run on 4-20% gradient gels, blotted and probed with polyclonal antibodies specific for Tau or GFAP, respectively. The amounts of proteins loaded for the human recombinant proteins (Hu Rec) or the PBMC extracts (CTE and CL) are shown above. The arrows indicate the position of bands, with the thick arrows pointing to the major full-length non-aggregated proteins.

FIG. 4 shows fluorescence analysis of PBMC cells treated with antibodies for CD14 (red) and GFAP (green) (DAPI stain not shown). A total of 1765 cells (ROls) were analyzed and ordered first by the mean green fluorescence (left scale) of pixel clusters inside each ROI, and then by the mean red fluorescence (right scale) of those pixel clusters. By raising the threshold for mean green fluorescence intensity per cluster to 3000 OD units (right graph) four groups of cells became apparent. The first 1393 ROls (groups A and B) had no significant red fluorescence, while group B had 15 ROls (0.8%) with green fluorescence exceeding the threshold. Groups C and D had significant red fluorescence but only group D (33 ROls, 1.9%) had a mean green fluorescence above the threshold.

FIG. 5 shows the results of single cell analysis testing for GFAP in PBMCs obtained from rats having been subjected to brain surgery (without electrode implantation).

FIG. 6 shows the results of single cell analysis testing for GFAP in PBMCs obtained from rats having been subjected to brain surgery with electrode implantation.

FIG. 7 shows GFAP fluorescent imaging and cell counts. Three image planes (GFAP1, GFAP2, GFAP3) from one sample were imaged at 5x and fluorescent cells (DAPI[350]/CD14[568]/GFAP[488]) were counted using ImageJ software. Cell counts were fairly high (low thousands). This sample contained on average 10.8% CD14+cells and of those cells roughly 1.7% were GFAP+(0.19% of total). There were a few cells which were GFAP+/CD14-.

FIG. 8 shows Tau fluorescent imaging and cell counts. Three image planes (Tau1, Tau2, Tau3) from one sample were imaged at 5x and fluorescent cells (DAPI[350]/CD14[568]/TAU[488]) were counted using ImageJ software. Cell counts were somewhat lower (due to division of sample between several slides for troubleshooting). This sample contained on average 8.4% CD14+cells and of those cells roughly 8.7% were TAU+(0.7% of total). There were a few cells which were TAU+/CD14−.

### DETAILED DESCRIPTION OF THE INVENTION

As previously discussed, the presence and/or amount of central nervous system (CNS)-derived debris may be useful for determining various states of the brain or CNS tissue and biological changes in the brain or CNS tissue, such as those associated with active central nervous system tissue damage, active central nervous system repair, active neurodegeneration, normal CNS processes, aging, etc. Thus, obtaining these neural-derived circulating phagocytes (that were previously in the central nervous system) can be used for monitoring a brain condition or neurological disease (e.g., monitoring worsening or improvement of a particular brain condition or neurological disease), detecting neurological damage (e.g., neurological damage associated with a disease or injury), detecting active neurodegenerative diseases, active central nervous system tissue damage, and/or active central nervous system repair, monitoring aging processes, monitoring normal CNS processes, etc.

As a non-limiting example, the methods herein include methods for preparing and/or analyzing CNS-derived compounds (obtained from outside the CNS). The methods may comprise isolating and/or sorting circulating phagocytes (e.g., peripheral circulating phagocytes) from a sample (e.g., fluid sample) obtained from outside central nervous system (CNS) tissue of a subject, e.g., blood, CSF, etc. The method may further comprise extracting lysate from the circulating phagocytes. The methods may further comprise analyzing the CNS-derived compounds in the lysate. The method may further comprise producing a fraction of the lysate and analyzing the CNS-derived compounds in the lysate fraction. In some embodiments, the method comprises producing a fraction of the lysate by selectively collecting CNS-derived compounds (e.g., specific CNS-derived compounds, e.g., biomarkers as described herein) and subsequently analyzing the CNS-derived compounds in the fraction.

In some embodiments, if the CNS-derived compound is displayed on the cell surface of the phagocytes, the method may comprise extracting circulating phagocytes from the sample (e.g., fluid sample) obtained from outside central nervous system (CNS) tissue of the subject and producing a fraction of the circulating phagocytes extracted by separating the phagocytes with membrane-bound CNS-derived peptides/compounds from the phagocytes without membrane-bound CNS-derived peptides/compounds.

As a non-limiting example, the methods herein include methods for preparing and/or analyzing CNS-derived compounds obtained from outside the central nervous system (CNS). The methods may comprise lysing a whole fluid sample obtained from outside central nervous system (CNS) tissue of a subject, e.g., lysing whole blood. The methods may further comprise analyzing the CNS-derived compounds in the lysed sample (e.g. lysed whole blood). The method may further comprise producing a fraction of the lysate and analyzing the CNS-derived compounds in the fraction. The method may comprise producing a fraction by selectively collecting CNS-derived compounds (e.g., biomarkers as described herein) and subsequently analyzing the CNS-derived compounds in the fraction.

As a non-limiting example, the methods herein include methods for preparing and/or analyzing CNS-derived compounds. The methods may comprise sorting and/or isolating circulating phagocytes (e.g., peripheral circulating phagocytes) from a sample obtained from outside central nervous system (CNS) tissue. The method may comprise simultaneously analyzing the CNS-derived compounds (the fraction comprises CNS-derived compounds, e.g., specific CNS-derived compounds, e.g., biomarkers as described herein). The methods herein may further comprise analyzing the CNS-derived compounds in the fraction.

As used herein, a patient or subject may refer to a human or an animal. An animal may include but is not limited to a mammal. Mammals may include but are not limited to primates (e.g., a human), a mouse, a rat, a llama, a rabbit, a dog, a primate, a guinea pig, a cat, a hamster, a pig, a goat, a horse, or a cow. The present invention is not limited to the aforementioned subjects or patients.

As used herein, the term “peripheral” refers to anything outside of brain tissue. For example, a peripheral phagocyte may be found in tissues outside of the brain or and/or fluids in the body, for example in blood, peripheral blood mononuclear cells (PBMCs), synovial fluid, cerebrospinal fluid (CSF), central nervous system tissues, synovial fluid, cystic fluid, lymph fluid, ascites, pleural effusion, interstitial fluid, ocular fluids, vitreal fluid, urine the like, or a combination thereof.

As such, samples herein include but are not limited to blood samples, CSF, tissue, or other appropriate samples that comprise CNS-related fluid and/or tissue. In some embodiments, the sample is blood, synovial fluid, cerebrospinal fluid (CSF), synovial fluid, cystic fluid, lymph fluid, ascites, pleural effusion, interstitial fluid, ocular fluids, vitreal fluid, urine, or a combination thereof.

Samples may be collected and processed and/or stored. In some embodiments, the container for the sample, e.g., the blood sample, comprises an anticoagulant. In some embodiments, the anticoagulant comprises citrate, heparin, or a combination thereof.

Phagocytes may include but are not limited to monocytes, macrophages, dendritic cells, granulocytes (e.g., neutrophils), lymphocytes, etc., and combinations thereof.

The methods herein may further comprise introducing to the sample a molecule for inhibiting degradation (or further degradation) of the neural-derived compound (e.g., CNS-derived compound, CNS-derived debris, etc.) in or on the phagocytes. For example, generally, any component that increases the pH of the phagolysosomes, which would inhibit the enzymes in the phagolysosomes, may help reduce the degradation of peptides (e.g., the biomarkers of interest) in the phagolysosomes. In some embodiments, the molecule for inhibiting further degradation of the neural-derived biomarker in the phagolysosome of the phagocytes comprises one or a combination of phagolysosomal protease inhibitors. In some embodiments, the protease inhibitor comprises leupeptin. In some embodiments, the molecule for inhibiting further degradation of the neural-derived biomarker in the phagolysosome of the phagocytes comprises a molecule that increases the pH of the phagolysosomes of the phagocytes in the first fluid sample. In some embodiments, the molecule for increasing the pH of the phagolysosomes of the phagocytes in the first fluid sample comprises an alkaline buffer. Alkaline buffers are well known to one of ordinary skill in the art, e.g., chloroquin, carbonate/bicarbonate buffer, buffers of pH 9.2 or above, weak base buffers, quinine, etc. In some embodiments, both a phagolysosomal inhibitor and alkaline buffer are added. In some embodiments, a protease inhibitor is introduced to the sample within 1 minute, 2 minutes, 3 minutes, 4 minutes, or 5 minutes, or within 10 minutes, 15 minutes, 20 minutes, etc., of when the sample is obtained.

The phagocytes may be obtained, collected, concentrated, etc. via a variety of means. For example, methods may feature a cell-affinity chromatography system herein the phagocytes interact with and/or bind a ligand immobilized on a system such as a filter, a membrane, a slide, a column, etc. The phagocytes may then be eluted after being captured by the chromatography system. In certain embodiments, the ligand is an antibody that is specific for the cell type of interest, e.g. the phagocyte. As a non-limiting example, the chromatography system may feature a spin column with a resin displaying a phagocyte-specific antibody, wherein the sample (e.g., blood) is introduced to the spin column. In some embodiments, the system features a slide displaying a phagocyte-specific antibody, wherein the sample (e.g., blood) is introduced to the slide. In some embodiments, the system features a syringe with a membrane displaying a phagocyte-specific antibody, wherein the sample (e.g., blood) is introduced to the syringe. In some embodiments, the method comprises introducing magnetic beads to the sample, whereupon phagocytes engulf the magnetic beads, yielding magnetic phagocytes. The method may further comprise separating the magnetic phagocytes using a magnetic separation mechanism. In some embodiments, the method comprises introducing to the sample a stimulator to stimulate phagocytosis of the magnetic beads by the phagocytes. In some embodiments, the magnetic beads are conjugated with an acid hydrolase inhibitor. In some embodiments, the magnetic beads are conjugated with an antibody or antibody component to stimulate phagocytosis. In some embodiments, the magnetic beads are introduced to the first fluid sample within 1 minute, 2 minutes, 3 minutes, 4 minutes, 5 minutes, 10 minutes, 15 minutes, or 20 minutes of when the first fluid sample is obtained. In some embodiments, the magnetic beads/particles are coated with a phagolysosomal inhibitor (e.g., leupeptin). In some embodiments, the magnetic beads/particles are coated with a mix of compounds, e.g., a phagolysosomal inhibitor (e.g., leupeptin), an antibody (e.g., IgG, IgG(Fc), etc.).

In some embodiments, the magnetic separation mechanism comprises a magnetic column or magnetic rack. In some embodiments, the container (for the blood sample) comprises Ficoll. In some embodiments, the container (for the blood sample) does not comprise Ficoll or is free of Ficoll. In some embodiments, the magnetic phagocytes are separated using the magnetic separation mechanism within 1 hour of harvesting of the first fluid sample. In some embodiments, the magnetic phagocytes are separated using the magnetic separation mechanism within 12 hours of harvesting of the first fluid sample. In some embodiments, the magnetic phagocytes are separated using the magnetic separation mechanism within 24 hours of harvesting of the first fluid sample. In some embodiments, the magnetic phagocytes are separated using the magnetic separation mechanism within 48 hours of harvesting of the first fluid sample. In some embodiments, the magnetic phagocytes are separated using the magnetic separation mechanism after the sample has been stored for a period of time.

As described above, in some embodiments, the methods herein may comprise subjecting the sample fluorescence-activated cell sorting (FACS). Fluorescence-activated cell sorting (FACS) is a type of flow cytometry that sorts a mixture of biological cells, one at a time, into separate containers based upon the specific light scattering and fluorescent characteristics of each cell. It provides quantitative recording of fluorescent signals from individual cells as well as physical separation of cells of particular interest. Generally, a current of a rapidly flowing stream of liquid carries a suspension of cells through a nozzle. The flow is selected such that there is a large separation between cells relative to their diameter. Vibrations at the tip of the nozzle cause the stream of cells to break into individual droplets, and the system is adjusted so that there is a low probability of more than one cell being in a droplet. A monochromatic laser beam illuminates the droplets, which are electronically monitored by fluorescent detectors. The droplets that emit the proper fluorescent wavelengths are electrically charged between deflection plates in order to be sorted into collection tubes.

The present invention is not limited to fluorescent assays, e.g., fluorescent microscopy or imaging. In some embodiments, the methods herein comprise colorimetric assays. As a non-limiting example, the methods may comprise a colorimetric ELISA. In some embodiments, the methods herein comprise imaging without a microscope. In some embodiments, the methods herein comprise using an image analysis system, wherein images may be obtained from surfaces such as a slide or a plate (e.g., microplate well), etc.

In some embodiments, the methods herein comprise sorting the phagocytes using a magnetic mechanism, e.g., magnetic extraction.

In some embodiments, the phagocytes are stained with a labeled phagocyte-specific binding moiety. In some embodiments, a target biomarker, e.g., a CNS-derived biomarker inside or on the surface of phagocytes) is stained with a different color (a second color) than the phagocyte-specific binding moiety (a first color). The methods may further comprise measuring a ratio of the first color to the second color, wherein the ratio of colors is indicative of an amount of target biomarker molecules inside or displayed on the cell surface of said phagocytes.

In some embodiments, the circulating phagocytes have a specific immunotype. In some embodiments, the circulating phagocytes are concentrated. In some embodiments, the circulating phagocytes are concentrated based on immunotype.

The phagocytes containing the biomarkers of interest may be characterized and/or isolated and/or concentrated based on immunophenotyping. This process may be used for investigative purposes, for example to help determine if there is a subpopulation of cells with the particular biomarker of interest. Further, the process, once a particular immunophenotype of cells is identified for a biomarker of interest, may be used as a technique for concentrating the phagocytes during sample preparation and analysis. The association of a particular immunophenotype cell and a biomarker of interest may be achieved by any appropriate method, e.g., flow cytometry, immunofluorescence microscopy, etc. The results may identify known phagocytic cell types (CD14+ monocytes and/or macrophages (CD 68/CD11b), CD15+/CD66b+ neutrophils, CD15+/CD66b+/MHC II+ neutrophils,) to be the source of particular biomarkers (e.g., neural antigens) in PBMCs.

Non-limiting examples of lineage antigens for immunophenotyping and immunoselection may include CD14, CD16, CD71, CD11a, CD11b, CD11c, CD15^{low}, CD33, CD64, CD68, CD80, CD86, CD105/endoglin, CD115, CD163, CD195/CCR5, CD282/TLR2, CD284/TLR4, HLA-DR/MHC Class II, ILT1, ILT3, ILT4, ILT5, Mature Macrophage Marker^{surface}, CD1a, CD1b, CD1c, CD40, CD49d, CD83, CD85g/ILT7^{pDC}, CD123, CD197/CCR7, CD205/DEC-205, CD207/Langerin, CD209/DC-SIGN, CD273/B7-DC, CD289/TLR9, CD303, CD304, CMKLR-1^{pDC}, the like, or a combination thereof. The present invention is not limited to the aforementioned antigens. Further, the lineage antigens are not limited to human antigens and may include any appropriate corresponding cellular antigen in a different species. For example, in some embodiments, the subject or animal is a mouse, and the lineage antigens may include but are not limited to CD11a, CD11b, CD13, CD14^{mono}, CD16/CD32, CD64, CD68, CD80, CD86, CD107/Mac3, CD115, CD282/TLR2, CD284/TLR4, F4/80, Galactin-3/Mac-2, GITRL, MHC Class II, 33D1, CD4, CD8, CD11b^{low}, CD11c, CD40, CD45R/B220^{pDC}, CD83, CD123^{pDC}, CD197/CCR7, CD205/DEC-205, CD207/Langerin, CD209/DC-SIGN^{immature}, CD273/B7-DC, CD289/TLR9, CD317/PDCA-1^{pDC}, F4/80^{low}, MHC Class II, Siglec H^{pDC}, the like, or a combination thereof. Human macrophage/monocyte markers include but are not limited to: CD11a, CD11b, CD11c, CD14, CD15^{low}, CD33, CD64, CD68, CD80, CD86, CD105/endoglin, CD115, CD163, CD195/CCR5, CD282/TLR2, CD284/TLR4, HLA-DR/MHC Class II, ILT1, ILT3, ILT4, ILT5, and Mature Macrophage Marker^{surface}. Human dendritic cell markers include but are not limited to: CD1a, CD1b, CD1c, CD11c, CD14, CD40, CD49d, CD 80, CD83, CD85g/ILT7^{pDC}, CD123^{pDC}, CD197/CCR7, CD205/DEC-205, CD207/Langerin, CD209/DC-SIGN, CD273/B7-DC, CD289TLR9, CD303, CD304, CMKLR-1^{pDC}, and HLA-DR/MHC Class II, Mouse macrophage/monocyte markers include but are not limited to: CD11a, CD11b, CD13, CD14^{mono}, CD16/CD32, CD64, CD68, CD80, CD86, CD107/Mac3, CD115, CD282/TLR2, CD284/TLR4, F4/80, Galactin-3/Mac-2, GITRL, and MHC Class II. Mouse dendritic cell markers include but are not limited to: 33D1, CD4, CD8, CD11b^{low}, CD11c, CD40, CD45R/B220^{pDC}, CD80, CD83, CD86, CD123^{pDC}, CD197/CCR7, CD205/DEC-205, CD207/Langerin, CD209/DC-SIGN^{immature}, CD273/B7-DC, CD289/TLR9, CD317/PDCA-1 ^{PDC}, F4/80^{low}, MHC Class II, and Siglec^{pDC}.

The methods herein may also comprise introducing a factor or combination of factors to the sample and/or the phagocytes and/or the fraction, wherein the fraction helps prevent apoptosis of the phagocytes. Non-limiting examples of factors that may be introduced include epidermal growth factor (EGF), fetal bovine serum (FBS), other growth factors, a nutrient-rich medium, etc.

The present invention also features methods for preservation of samples for preserving the amount and/or structure and/or location of the CNS-derived biomarker(s) of interest (e.g., for preserving the amount and/or structure and/or location of the epitope(s) of interest). For example, the present invention provides methods for treating samples for the purposes of preserving the biomarker, e.g., via heat denaturation (wherein proteolytic enzymes or other factors are inhibited without affecting the biomarker, e.g., the epitope of the biomarker, to a large extent). Other methods of preservation may include freeze drying or other rapid freezing processes, application of heparin or other factors, modifying the pH of the sample, etc. The present invention is not limited to the aforementioned methods or compositions.

In some embodiments, the phagocytes obtained from the sample are permeabilized. In some embodiments, the phagocytes are lysed via various means, e.g., hypotonic solution treatment, detergent solution treatment, mechanical stress, etc.

### Biomarkers

Various neural-derived debris antigens or biomarkers may be found in circulating/recirculating (peripheral) phagocytes in the peripheral blood.

Without wishing to limit the present invention to any theory or mechanism, it is believed that in certain situations, the CNS-derived compounds (e.g., debris from brain tissue or other central nervous system tissue) may be compounds that would not be found outside of the CNS tissue or would not be found at particular levels outside the CNS tissue unless, for example, trauma had occurred, a disease process had been active, a disease process is currently active or is about to become active, etc. The present invention is not limited to the presence of the biomarkers (or the presence of the biomarkers at particular levels) is only related to disease or trauma. In some embodiments, the presence of the CNS-derived compounds (or the presence of the CNS-derived compounds at particular levels) is related to an aging process. In some embodiments, the presence of the CNS-derived compounds (or the presence of the CNS-derived compounds at particular levels) is related to a normal CNS process. Subjects considered to be “normal” (e.g., those showing normal neurological functions, e.g., as determined by a qualified healthcare provider) may have detectable levels of the CNS-derived compound(s). The present invention allows for the analysis of the levels of the CNS-derived compounds relative to a patient's baseline level, e.g., a level of the compounds at an earlier time point. Referring to the detection of the CNS-derived compounds, in some embodiments, the compounds may be found to be higher than a predetermined threshold (e.g., a patient's standard or baseline level, an industry standard, a laboratory standard, etc.). In some embodiments, the compounds are found to be lower than a predetermined threshold (e.g., a patient's standard or baseline level, an industry standard, a laboratory standard, etc.).

For any of the embodiments herein, the CNS-derived compound or antigen may be one or more of the following compounds: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2 (tyrosine 3-monooxygenase etc.), a synaptic antigen (e.g., PSD-95 protein, neurogranin, SNAP-25, TDP-43, etc.), transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin All, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), a breakdown product thereof, a fragment or fragments thereof, etc. The present invention is not limited to the aforementioned biomarkers or antigens.

As a non-limiting example, neuromelanin may be measured in several ways, e.g., via the binding of labeled melanin selective peptides (e.g., 4B4 peptide), e.g., biotinylated 4B4 peptide; a control peptide P601G may be used as a control); the binding of monoclonal or polyclonal antibodies to melanin; measurement of metal binding to melanin; measurement of the semiconductor properties of melanin; measurement of the fluorescence properties of melanin; and extraction of melanin from recirculating phagocytes and subsequent quantification of melanin, it's components or adducts (both natural or synthetic); physical methods such as gas chromatography, liquid chromatography or mass spectrometry; and combinations of these methods.

The term Tau biomarker may refer to a particular epitope of Tau, e.g., an epitope within a particular region of amino acids. In some embodiments, the epitope is in a region from aa 240-441. In some embodiments, the epitope is in a region from aa 243-441. In some embodiments, the epitope is in a region from aa 244-274. In some embodiments, the epitope is in a region from aa 275-305. In some embodiments, the epitope is in a region from aa 306-336. In some embodiments, the epitope is in a region from aa 337-368. In some embodiments, the epitope is in a region from aa 388-411. The present invention is not limited to these regions.

Further, the epitope may be in shorter regions of amino acids, e.g., aa 244-260, aa 270-280, aa 290-310, aa 330-360, etc.

As previously discussed, the biomarkers may refer to epitopes. For example, the biomarker may be an epitope of GFAP. As a non-limiting example, the epitope may be a GFAP epitope between amino acids 213-340, or a GFAP epitope between 119-178. The present invention is not limited to the aforementioned epitopes. The present invention is not limited to full-length biomarkers and includes epitopes for the biomarkers described herein.

In some embodiments, the biomarker is detected using an HPLC technique (e.g., HPLC-UV, HPLC-fluorescence), a luminescence technique, an immunoassay technique, a streptavidin/biotin technique, or a combination thereof. The present invention is not limited to any particular biomarker detection technique.

In some embodiments, a combination (e.g., a pair) of biomarker-specific antibodies are used for isolating and detecting the biomarker of interest. For example, an ELISA assay may use a first biomarker-specific antibody as a capturing antibody and a second biomarker-specific antibody as a detection antibody. The present invention is not limited to the use of any specific pair of antibodies. The present invention includes a combination of any of the antibodies disclosed herein or antibodies specific to the biomarker of interest not necessarily listed herein, e.g., those that may be produced in the future, those that are commercially available, etc.

In some embodiments, the biomarker (neural-derived debris, antigen, etc.) is an intracellular component. In some embodiments, the biomarker is a membrane-bound component. In some embodiments, more than one biomarker is detected in the sample(s).

The biomarker may be of various lengths. For example, in some embodiments, the biomarker is from 5 to 20 amino acids. In some embodiments, the biomarker is from 20 to 40 amino acids. In some embodiments, the biomarker is from 40 to 80 amino acids. In some embodiments, the biomarker is from 80 to 150 amino acids. In some embodiments, the biomarker is from 150 to 200 amino acids. In some embodiments, the biomarker is from 200 to 300 amino acids. In some embodiments, the biomarker is from 300 to 400 amino acids. In some embodiments, the biomarker is from 400 to 500 amino acids. In some embodiments, the biomarker is from 500 to 600 amino acids.

The biomarker may comprise various regions of the full-length protein. For example, in some embodiments, the biomarker comprises the amino-terminus (e.g., N-terminus, NH2-terminus, N-terminal end, amine-terminus). The amino-terminus refers to the amino acid at the end of a protein or polypeptide that has a free amine group (—NH2). In some embodiments, the biomarker comprises about the first 15 amino acids. In some embodiments, the biomarker comprises about the first 25 amino acids. In some embodiments, the biomarker comprises about the first 50 amino acids. In some embodiments, the biomarker comprises about the first 75 amino acids. In some embodiments, the biomarker comprises about the first 100 amino acids. In some embodiments, the biomarker comprises about the first 125 amino acids. In some embodiments, the biomarker or fragment thereof comprises the carboxy-terminus (e.g., C-terminus, COOH-terminus, C-terminal end, carboxyl-terminus). The carboxy-terminus refers to the amino acid at the end of a protein or polypeptide that has a free carboxylic acid group (—COOH). In some embodiments, the biomarker comprises the last 100 amino acids.

In some embodiments, the step of detecting the biomarker in the sample comprises subjecting the sample to a western blot, an enzyme-linked immunosorbent assay (ELISA), a lateral flow assay, a radioimmunoassay, an immunohistochemistry assay, a bioluminescent assay, a chemiluminescent assay, a mass spectrometry assay, a flow cytometry assay (e.g., fluorescence-activated cell sorting (FACS)), or a combination thereof and the like. Such assays are well known in the art.

In some embodiments, the step of detecting the biomarker further comprises contacting the sample with an antibody that binds to the biomarker and detecting an antibody-biomarker complex. The step of detecting an antibody-biomarker complex may comprise subjecting the sample to a microarray, western blot, an enzyme-linked immunosorbent assay (ELISA), a lateral flow assay, a radioimmunoassay, an immunohistochemistry assay, a bioluminescent assay, a chemiluminescent assay, a flow cytometry assay (e.g., fluorescence-activated cell sorting (FACS)), fluorescence staining, or a combination thereof and the like. In some embodiments, detecting the antibody-biomarker complex indicates the presence of the particular disease or condition of investigation or a risk of the particular disease or condition of investigation.

As described above, in some embodiments, the step of detecting the biomarker may comprise subjecting the sample fluorescence-activated cell sorting (FACS). Fluorescence-activated cell sorting (FACS) is a type of flow cytometry that sorts a mixture of biological cells, one at a time, into separate containers based upon the specific light scattering and fluorescent characteristics of each cell. It provides quantitative recording of fluorescent signals from individual cells as well as physical separation of cells of particular interest. Generally, a current of a rapidly flowing stream of liquid carries a suspension of cells through a nozzle. The flow is selected such that there is a large separation between cells relative to their diameter. Vibrations at the tip of the nozzle cause the stream of cells to break into individual droplets, and the system is adjusted so that there is a low probability of more than one cell being in a droplet. A monochromatic laser beam illuminates the droplets, which are electronically monitored by fluorescent detectors. The droplets that emit the proper fluorescent wavelengths are electrically charged between deflection plates in order to be sorted into collection tubes.

### Kits

The present invention also features kits comprising reagents or tools for performing the methods described herein. For example, in some embodiments, the kit comprises sample collection tubes. In some embodiments, the kit comprises biomarker-specific antibodies and/or phagocyte-specific antibodies. In some embodiments, the kit comprises secondary antibodies. In some embodiments, the kit comprises reagents such as columns, magnetic beads, nanoparticles, etc.

In some embodiments, the kits further comprise reagents for preserving the sample, e.g., for preserving the amount, structure, and/or location of neural-derived cargo.

The kit may further comprise other appropriate reagents, manuals, equipment, etc. For example, the kit may comprise reagents for automated assays. In some embodiments, the kit comprises reagents for multiplex assays.

### Validating Induction of Animal or Clinical Models and Usefulness of Drugs or Treatments

The present invention also provides methods for validating animal or clinical models, e.g., the process of inducing an animal or clinical model.

For example, the present invention provides a method of validating a model for a neurodegenerative disease or condition in an animal. The method may comprise introducing an induction (e.g., drug, agent, physical change, genetic modification, trauma such as concussion, etc.) to the animal to cause a neurodegenerative disease or condition or phenotype thereof; isolating circulating phagocytes from a fluid sample from the animal at a time point after induction, the fluid sample being from outside of a brain tissue of the animal; and detecting a level of a central nervous system damage-associated biomarker in the phagocytes. An abnormal level of the central nervous system damage-associated biomarker may be indicative of presence of a neurodegenerative disease or condition, which may thereby validate the animal is a model for a neurodegenerative disease or condition.

In some embodiments, the induction is overexpression of a gene, e.g., in a portion of the brain/CNS tissue or at least a portion of the brain/CNS tissue. As a non-limiting example, the induction may be overexpression of neuromelanin. In some embodiments, the brain tissue is the substantia nigra.

In some embodiments, the fluid sample is a blood sample. In some embodiments, the time point is from 5 to 30 days. In some embodiments, the time point is from 21 to 60 days. In some embodiments, the time point is from 1-4 months. In some embodiments, the time point is at least one week. In some embodiments, the animal is a mouse or rat. In some embodiments, the animal is a primate.

The present invention also provides methods for validating the usefulness of drugs or treatments, e.g., methods for validating usefulness of a drug or composition or treatment for treating central nervous system tissue damage, central nervous system repair, or neurodegeneration. The present invention also provides methods for defining a therapeutic window.

For example, in some embodiments, the method of validating usefulness of a drug or composition or treatment for treating central nervous system tissue damage, central nervous system repair, or neurodegeneration comprises administering the drug or composition or treatment to a subject having or suspected of having central nervous system tissue damage, central nervous system repair, or neurodegeneration; isolating circulating phagocytes from a fluid sample from the subject at a time point after administration of the drug or composition (wherein the fluid sample is from outside of a brain tissue of the subject); and detecting a level of a central nervous system damage-associated biomarker in the phagocytes. In some embodiments, an abnormal level of the central nervous system damage-associated biomarker relative to a control validates the usefulness of the drug for treating central nervous system tissue damage, central nervous system repair, or neurodegeneration.

In some embodiments, the subject is an animal model for central nervous system tissue damage, central nervous system repair, or neurodegeneration. In some embodiments, the time point is from 5 to 30 days. In some embodiments, the time point is from 21 to 60 days. In some embodiments, the time point is from 1-4 months. In some embodiments, the time point is at least one week. In some embodiments, the subject is a human. In some embodiments, the subject is a mouse or rat. In some embodiments, the subject is a primate. In some embodiments, the method is for determining a therapeutic window of the drug or agent or treatment.

The methods of validating animal models are not limited to the aforementioned examples. For example, any of the methods herein may be used to analyze the CNS-derived biomarkers.

### EXAMPLE 1

The following describes an example of a method of the present invention. The present invention is not limited to the methods or materials described herein. For example, the present invention is not limited to cell preparation tubes (CPTs) and includes alternative collection vessels and methods.

A laboratory receives a patient's blood sample collected in a CPT tube. PBMCs are obtained from a BD Vacutainer™ CPT tube using a cell separation procedure. The cells are washed three times in 1X PBS and centrifuged in a horizontal rotor (swing-out head) for a minimum of 5 minutes at 1200 to 1500 RCF (Relative Centrifugal force). The supernatant is removed, and the cells are resuspended in 1X PBS. After the final wash, extracts of the PBMCs are prepared by lysing with a hypotonic solution or other method. Then the lysate is subjected to assay involving an antibody that binds to Tau, e.g., a protein fragment comprising the phosphorylated serine residue Ser-404.

### EXAMPLE 2

The following describes an example of a method of the present invention. The present invention is not limited to the methods or materials described herein.

PBMCs are obtained from a BD VacutainerTM CPT tube using a cell separation procedure. The cells are washed three times in 1X PBS and centrifuged in a horizontal rotor (swing-out head) for a minimum of 5 minutes at 1200 to 1500 RCF (Relative Centrifugal force). The supernatant is removed, and the cells are resuspended in 1X PBS. After the final wash, the cells are resuspended to approximately 4.0 mL in 1X PBS. Approximately 50 μL of the cell suspension to be analyzed is transferred into tubes for double staining with selected antibody pairs. Ten pL of 40 mg/mL normal human IgG (Sigma-Aldrich) for a total of 400 μg is added to each tube to block FC binding. The appropriate cell surface monoclonal antibodies CD3 PE, CD19 PE or CD14 PE are added at this time and incubated for 20 minutes at room temperature.

One hundred μl of Dako Intrastain™ Reagent A (fixative) is added to each tube and then mixed gently with a vortex mixer to ensure that the cells are in suspension. Cells are incubated at room temperature for 15 minutes. Two mL of 1X PBS working solution is added to each test tube and mixed gently. The tubes are centrifuged at 300 X g for 5 minutes. Supernatant is aspirated leaving about 50 μl of fluid. The fluid is mixed thoroughly to ensure that the cells are in suspension.

One hundred pL of Dako Intrastain™ Reagent B (permeabilization) is added to each tube. The appropriate amount of the antibody specific for the multiple sclerosis-associated antigen is added to the appropriate tubes. The tubes are mixed gently to ensure that the cells are in suspension and incubated at room temperature for 15-60 minutes. Two mL of 1X PBS working solution is added to each test tube and mixed gently. The tubes are centrifuged at 300 X g for 5 minutes, and then the supernatant is aspirated, leaving approximately 50 μl of fluid. The fluid is mixed thoroughly to ensure that the cells are in suspension.

One hundred μL of Dako Intrastain™ Reagent B (permeabilization) is added to each tube. The appropriate volume of the 2nd step antibody conjugated to FITC (specific to the multiple sclerosis-associated antigen) is added to the appropriate tubes. The tubes are mixed gently to ensure that the cells are in suspension and incubated at room temperature for 15-60 minutes. To each tube, 2.0 mLs of 1XPBS working solution is added. The tubes are mixed gently then centrifuged at 300 X g for 5 minutes. The supernatant is aspirated, leaving approximately 50 μl of fluid. The tubes are mixed thoroughly to ensure that the cells are in suspension.

The pellet is resuspended in an appropriate volume of fluid for flow cytometry analysis. The sample is analyzed on a flow cytometer within 24 -48 hours. For analysis, the gate is on the monocyte population and the data is collected in list mode. Qualitative and or quantitative differences are determined between normal and MS patients using the analysis software. Optimization steps include varying incubation time with antibodies, fixation time and permeabilization time.

### EXAMPLE 3

The following describes examples of instructions for receiving, handling, processing and storage of incoming blood samples for fluorescent microscopy imaging of human tau. This procedure may be used when a blood sample is received for fluorescent imaging of tau protein within peripheral blood mononuclear cells. The present invention is not limited to the methods or materials described herein.

The present invention is not limited to CPTs (cell processing tubes); in some embodiments, other systems or samples (or sample fractions) may be used such as heparin, whole blood, etc. Note that in this example, PBMC refers to Peripheral Blood Mononuclear Cells, CPT refers to Cell Processing Tube, RCF refers to Relative Centrifugal Force, Plasma refers to the fluid portion of whole blood in which the particulate components are suspended (in samples, contains anticoagulant to retain clotting factors), and the RPM Speed

$$Setting = \sqrt{\frac{RCF \times 100 , 000}{1.12 \times r}} .$$

Equipment needed: Centrifuge, 15 mL conical tube, Freezer (−80° C.), Microfuge plastic aliquot vials, Aluminum foil, Frosted microscope slides, 22×50 mm coverslips.

Reagents needed: PBS—Phosphate Buffered Saline (pH 7.4), RBC lysis buffer (ACK), BSA—Bovine Serum Albumin (1% in PBS with 0.1% sodium azide (NaN₃)), Anti-CD14-Texas Red, 10% buffered formalin (1:10 dilution of 37% formaldehyde in PBS), DAPI—4′,6-diamidino-2-phenylindole (300 nM), Dako Permeabilization Reagent B, Rabbit anti-hTau IgG, Goat serum (1% in PBS), Anti-rabbit IgG-FITC, Diamond antifade mountant, Nail polish.

### Plasma and PBMC Harvesting Procedures

Blood is collected into a citrate CPT (Becton Dickinson). A single 8 mL or 4 mL tube per subject may be received. Samples should be filled to capacity. Sample tubes undergo initial centrifugation the same day they are collected, and immediately after being drawn if possible. The centrifugation is for 30 minutes at 1500-1800 RCF (3000 rpm in a 17 cm diameter centrifuge). Store the spun tubes at 2-8° C. until ready to complete processing. PBMCs shall be harvested within 36 hours of the blood draw. Harvest plasma from the upper portion of the top layer, avoiding the Ficoll and cell layer near the gel plug, collecting up to 1 mL per 4 mL sample. Plasma is typically collected into 25-250 μL aliquots in plastic microfuge vials. Store at −80° C.

Pour the remaining fluid into a 15 mL conical tube, and add lx PBS to mostly fill the tube. If processing multiple samples from the same subject, the samples may be pooled into a single 15 mL tube. Centrifuge for 20 minutes at 300 RCF (1200 rpm in a 17 cm diameter centrifuge). Pour off and discard the supernatant, using a thin steady stream to avoid disturbing the pellet, or aspirate with a vacuum.

Add 3-5 mL of RBC lysis buffer. Resuspend the pellet by gently vortexing or tapping the tube with the index finger. Incubate for 5 minutes. Add PBS to mostly fill the tube. Centrifuge for 20 minutes at 300 RCF then aspirate the supernatant.

### PBMC Staining Procedures

Dilute anti-CD14-Texas Red 1:50 in 1% BSA-PBS with 0.1% NaN₃. Add 100 μL of this dilution and resuspend the pellet. Let sit for 30 minutes protected from light by wrapping the tube in aluminum foil. The cells should remain protected from light from this point forward. Wash the cells by adding PBS to mostly fill the tube then centrifuging for 20 minutes at 300 RCF then aspirating the supernatant.

Add 100pL 10% buffered formalin and 100 μL DAPI (300 nM) and resuspend the pellet. Let sit for 15 minutes. Wash the cells by adding PBS to mostly fill the tube then centrifuging for 20 minutes at 300 RCF then aspirating the supernatant.

Add 100 μL Dako Permeabilization Reagent B and resuspend the cells. Let sit for 15 minutessit 15 minutes. Wash the cells by adding PBS to mostly fill the tube then centrifuging for 20 minutes at 300 RCF then aspirating the supernatant.

Dilute rabbit anti-hTau 1:100 in 1% goat serum-PBS. Add 100 μL of this dilution and resuspend the pellet. Let sit for 15 minutes. Wash the cells by adding PBS to mostly fill the tube then centrifuging for 20 minutes at 300 RCF then aspirating the supernatant.

Dilute anti-rabbit IgG-FITC 1:100 in 1% goat serum-PBS. Add 100 μL of this dilution and resuspend the pellet. Let sit for 15 minutes. Wash the cells by adding PBS to mostly fill the tube then centrifuging for 20 minutes at 300 RCF then aspirating the supernatant.

Add 100pL 10% buffered formalin and resuspend the pellet. Let sit for 15 minutes. Cells may be stored at this point prior to slide preparation by refrigerating the foil-covered 15 mL conical tube.

Place a 20-30 μL drop of cells onto the surface of a glass microscope slide, followed by a 20 μL drop of antifade mountant. Apply a 22×50 mm coverslip and line the slip with nail polish to seal. Let the slide sit at least 20 minutes to dry then loosely cover in foil and refrigerate. Note: wrapping the slide too tightly with foil may result in nail polish smudging the surface of the slide, resulting in poor image quality.

### EXAMPLE 4

This following describes an example of an immunofluorescence protocol for suspension cells. The present invention is not limited to the methods or materials described herein.

### Cell Preparation for Suspension Cells

1. Centrifuge the cell suspension at 1,500 rpm for 5 min, discard supernatant. 2. Wash cells with 1 mL of 1X PBS and obtain a pellet by centrifugation at 1,500 rpm for 5 min.

### Fixation (Methanol as fixative)

1. Incubate the cells with 700 μL 100% ice-cold methanol for 5 min at −20° C. followed by centrifugation at 1,500 rpm for 5 min. 2.. Discard supernatant and mix thoroughly with 800 μL of 1X PBS and centrifuge at 1,500 rpm for 5 min.

### Permeabilization

1. Add 100 μL of Dako Permeabliization reagent B and resuspend. Incubate the cells at room temperature for 15 min followed by pelleting at 1,500 rpm for 5 min. 2. Discard the supernatant and add 800 μL of 1X PBS to the pellet, mix thoroughly and centrifuge at 1,500 rpm for 5 min.

### Blocking

1. Add 1 mL 2% BSA and 1% Goat serum in 1X PBS. Incubate the cells at room temperature for 60 min.

### Immunostaining

1. Add the desired concentration of primary antibody [1:50 anti-CD14-Texas Read, 1:100 anti-Tau-FITC, 1:100 anti-GFAP] diluted in 200 pL of 0.1% BSA 1% Goat serum to the cells and incubate for 3 hours at room temperature. 2. Remove primary antibody solution and wash the cells three times with 500 μL of 1X PBS. 3. Add 100uL desired concentration of fluorescent dye-labeled secondary antibody if necessary [1:100 anti-rabbit IgG-FITC in 1% Goat serum] and 100μL DAPI (300nM) diluted in 500 pL of 0.1% BSA and incubate for 45 min at room temperature protected from light. 4. Wash the cells three times with 500 μL of 1X PBS-T. 5. Note: Use extra tube for controls. 6. Control #1—without antibodies, only include counterstains. 7. Control #2—with fluorescent dye-labeled secondary antibody only, without including primary antibody to test for specificity of fluorescent staining and to avoid artifacts based on autofluorescence of the cells. 8. Single primary antibody stains to test for any interference between antibodies.

### Mounting

1. Add 20-40 uL drop of cells onto the surface of the glass microscope slide, allow to air dry. 2. Add 20-40 uL of antifade mountant. Apply a 22×50 mm coverslip and seal with nail polish. 3. Let dry for 20 minutes and store at 4° C.

### EXAMPLE 5

The following example describes a non-limiting procedure for pre-analytical processing of blood samples.

Equipment/Reagents Needed: Centrifuge; 50mL conical tube; Storage vials for aliquots, −80° C. capable, 100 uL-1 mL (e.g. polypropylene microcentrifuge tubes); PBS—Phosphate Buffered Saline, pH 7.4; RBC lysis buffer (ACK); Protease inhibitor (e.g. G Biosciences ProteaseArrest [100X]); Magnetic nanoparticles/beads.

Blood may be collected into an anticoagulant containing tube (Becton Dickinson) that also contains 1mg of magnetic nanoparticles. A single 8 mL or 4 mL tube per subject may be received. Samples may contain the minimum volume specified by Table 1 (below). Samples for research may be collected under an IRB protocol. Phagocytes may be harvested within 36 hours of the blood draw. After receipt of a sample, phagocytes are harvested by sedimentation on a magnet. While the phagocytes are held by the magnet, all other blood components may be poured or aspirated off. The phagocytes are then washed twice with PBS.

TABLE 1

| | |
|---|---|
| 8 mL CPT | 4 mL CPT |

| | | | | |
|---|---|---|---|---|
| Min Draw Volume | 6 | mL | 3 | mL |
| DI Water for lysing PBMC | 500 | uL | 250 | uL |
| 100x Protease inhibitor | 5 | uL | 2.5 | uL |
| Chloroquin (4M) | 5 | uL | 2.5 | uL |

Resuspend the pellet in the volume of deionized water specified by Table 1 to lyse the Phagocytes. If processing multiple tubes from the same subject and collection time, the pellets from two tubes may be recombined in the total volume specified for one tube.

Bring the magnetic nanoparticles to the side wall of the tube using the magnet then remove the cell lysate with a micropipette and place in a fresh microfuge tube. Add the volume of protease inhibitor specified by Table 1. Add the volume of Chloroquin specified by Table 1. Aliquot and store. Lysates are typically collected into 25-100 uL aliquots.

Lysates may be stored at −80° C., in a freezer monitored by an external monitor and labeled with the correct contact information to call if a failure is noted. Assays of lysates may be conducted within 30 days of harvesting. Sample preparations may be frozen at least 24 hours prior to assaying. Label primary container (box or conical tube containing aliquots) with sample identifier, type of sample (plasma or PBMC lysate), date processed, and processor's initials. After experimentation, samples may either be stored or destroyed according to the IRB protocol under which they were collected.

An alternative process includes the use of magnetic nanoparticles coated with leupeptin, magnetic nanoparticles coated with a mix of leupeptin and human IgG(Fc), etc.

In some embodiments, in lieu of chloroquin, a carbonate/bicarbonate buffer may be used. In some embodiments, in lieu of chloroquin any weak base may be used. In some embodiments, in lieu of or in addition to a base, leupeptin (e.g., 0.25 mM) may be added.

### EXAMPLE 6

The following describes methods, compositions, and applications of the present invention. The present invention is not limited to the methods or materials described herein.

Neuronal biomarkers can be useful for the diagnosis of brain trauma, dementia or disease, presenting the potential for early detection of neurodegeneration. But harmful metabolites are also generated in the healthy brain and are cleared through the glymphatic pathway. Glymphatic dysfunction may result in the accumulation of toxic proteins such as A-beta and Tau, leading to the invasion of phagocytes and subsequent neuroinflammation, thereby generating conditions prodromal for neurodegenerative diseases. Typically these molecules cannot spill directly into the bloodstream due to the action of the blood-brain barrier (BBB), but even when the BBB breaks down as a result of trauma or disease, it is possible that their concentration in serum or plasma may be near or below detection limits for standard enzyme-linked immunosorbent assays (ELISA). This limitation can be partially overcome by either testing cerebrospinal fluid (CSF) or through the use of very sophisticated and expensive equipment solutions. However, those approaches do not necessarily lend themselves to routine clinical applications.

Inventors were the first to produce evidence that phagocytic cells carrying brain biomarkers can be detected in peripheral blood, not only in patients with neurologic disease, but even in healthy donors. Building on preliminary ELISA data, the present invention includes using single cell analysis to test for various brain-specific biomarkers in phagocytic cells, determining the cell type most useful for this analysis, and developing a method for their isolation from small amounts of peripheral blood.

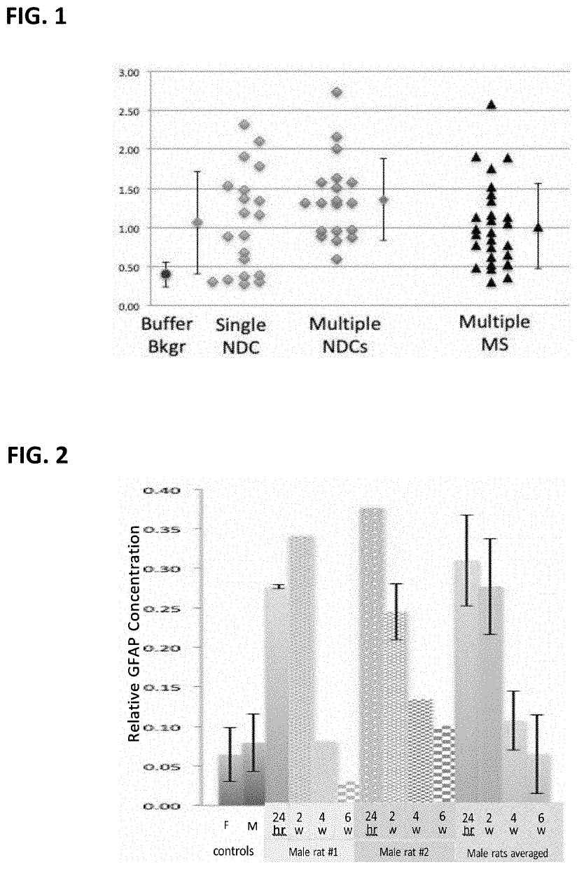

As an example of an ELISA assay, a single-sided enzyme linked immunosorbent assay was developed for glial fibrillary acid (GFAP), Tau, and neurofilament light (NFL) proteins. In short, purified human recombinant biomarker protein (for the standard curve) as well as whole PBMC extracts (samples) were diluted into buffer and adsorbed to microtiter wells and then probed with rabbit polyclonal antibodies specific for the biomarker protein, followed by an enzyme-linked secondary antibody specific for rabbit IgG. After removal of unbound antibodies and addition of substrate for the enzyme, the resulting color intensity was measured in a microplate reader. The equation for the linear trendline can be used to calculate the biomarker protein content in each sample, or normalized signal intensities can be used for comparison between different experiments as shown in FIG. 1. FIG. 1 shows the distribution of average Tau levels (normalized signal intensities) for MS patients and NDC controls. Blood samples were collected and tested over a period of 14 months with multiple intra-assay and inter-assay repeats for each sample. Surprisingly, the data do not show a difference between the MS population and normal controls. This may well be due to the fact that the MS patients were at different stages of disease and treatment and did not have an active brain tissue inflammation at the time of blood draw. Alternatively, a different set of antibodies is required to detect the biomarker molecules that have been enzymatically processed either before or after uptake by the macrophages. Also shown is the variation for a single healthy donor that was tested repeatedly over the same time period (Single NDC), revealing the same variation as the MS and NDC population.

Animal models of neurologic disorders are critical tools not only for the identification of new therapeutic targets or the development and testing of drugs and their efficacy in preclinical trials, but also to study the effect of novel physical treatment methodologies. An example is Deep Brain Stimulation, an established therapy for a variety of neurologic disorders, which involves the implantation of electrodes into the brain followed by electric stimulation. The mechanism of action and several side effects are not well understood and remain an active area of investigation, using predominantly mouse and rat models. The most obvious effect, destruction of neuronal tissue and the reaction of neighboring cells due to electrode implantation are typically studied by immunohistochemistry of brain sections with antibodies against GFAP, Tau or other neuronal biomarkers.

To test whether the effect of microelectrode implantation could be measured without having to sacrifice an animal, PBMC extracts were obtained from rats. FIG. 2 shows a significant increase of GFAP in the PBMCs of two male rats (M1 and M2) after electrode implantation, still detectable levels after 2 weeks of neurostimulation. The PBMC extracts of 2 female rats served as additional controls. Preliminary results point to the power of this technology, and its potential for the study of neurological effects resulting from brain manipulation and trauma.

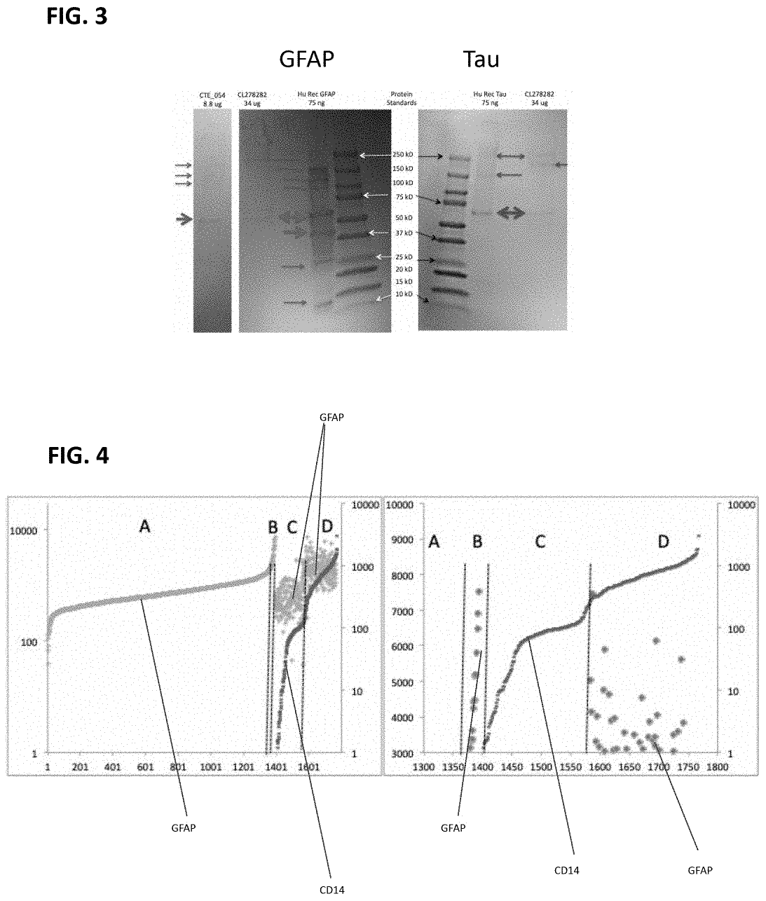

As an example of a western blot assay, purified human recombinant Tau and GFAP proteins as well as whole PBMC extracts were analyzed on Western blots probed with rabbit polyclonal antibodies to either Tau or GFAP and enzyme-linked secondary antibodies and substrate for the enzyme. As shown in FIG. 3, the recombinant Tau and the GFAP proteins are represented by multiple bands of different molecular weights (MW). The expected MW for both Tau and GFAP is ˜50 KD and bands with that approximate size can be seen in the extracts of the NDC control CL278282 as well as the suspected CTE patient CTE-054 in both blots. The higher MW bands are likely aggregation of Tau or GFAP, respectively, while the shorter bands are most likely breakdown products. Similar results were obtained when blots were probed with antibodies to NFL, confirming the presence of those proteins in the extract of the NDC control CL278282.

In order to measure the biomarker content of blood phagocytes, these cells are first isolated by ficoll gradients to separate peripheral blood monocytes (PBMC) from other blood components. The resulting cell mixture contains 10-20% monocytes, but only a small fraction of those may have visited the brain and then re-entered the bloodstream. The present invention is not limited to PBMCs or ficoll gradients.

The present invention also fluorescence microscopy (FM). Monocyte-specific cell surface proteins CD14 and CD16 allow their discrimination from other WBCs as well as identification of monocyte subpopulations. PBMC cells from a healthy donor were permeabilized and treated with differentially labeled antibodies specific for CD14 and either Tau or GFAP, as well as DAPI, a blue fluorophore that intercalates into DNA and specifically stains cell nuclei. Stained cells were affixed to a slide and analyzed by fluorescence microscopy. The results from three separate experiments are summarized in Table 2. Consistent with known cell distributions in human blood, approximately 10% of the nucleated PBMCs were found to be of monocyte origin (e.g., CD14 positive). Of those cells between 5% and 7% stained with the anti-biomarker antibody, suggesting the presence of both Tau and GFAP protein epitopes. Interestingly, an equal number of cells that did not stain with the CD14 antibody, which presumably were non-classical monocytes, dendritic cells, or perhaps neutrophils that were carried into the buffy coats, also carried a biomarker load. Note that this approach can provide for a rapid, highly sensitive diagnostic method that might be useful for point of care (POC) applications. (In Table 2, PBMCs were harvested from blood by Ficoll gradients and stained with DAPI, and fluorescently labeled antibodies for the macrophage surface marker CD14 and biomarkers Tau or GFAP; Cells were counted by an automated imaging system based on their differential stain.)

TABLE 2

| PBMCs (DAPI+) | Fraction of PBMCs | Fraction of PBMCs | Fraction of CD14+ | Fraction of CD14− |
|---|---|---|---|---|
| counted | that are CD14+ | that are GFAP+ | cells that are GFAP+ | cells that are GFAP+ |
| 15767 | 10.6% | 0.5% | 5.0% | 0.60% |

| PBMCs (DAPI+) | Fraction of PBMCs | Fraction of PBMCs | Fraction of CD14+ | Fraction of CD14− |
|---|---|---|---|---|
| counted | that are CD14+ | that are Tau+ | cells that are Tau+ | cells that are Tau+ |
| 11719 | 10.8% | 0.8% | 7.2% | 0.9% |

The demonstration by three different methods of detectable brain biomarkers in peripheral blood phagocytes—not only in rats after insertion of microelectrodes, but in the blood of human donors with suspected neurodegeneration, and in cognitively normal donors—presents the potential for a fundamentally novel approach to monitor brain health.

Phagocytic cells, including neutrophils, dendritic cells, and especially macrophages and microglia can play multiple roles in the inflammatory process leading to psychiatric and neurodegenerative disorders or metastatic brain tumors, as well as in their response to trauma or infectious agents. It is not surprising therefore that alterations in monocyte subset frequencies have been shown to be associated with altered clinical outcomes. Consequently, tools that enable the analysis of single blood-derived immune cells or cell components (exosomes or extracellular vesicles) have generated a significant amount of interest since they provide an alternate and perhaps unique source for biomarkers of clinical relevance. Differentiating monocyte and macrophage subsets with regard to their brain biomarker load may therefore be of significant importance for the diagnosis and potential therapy of neurological disease and is an integral part of the present invention.

One of the considerations with the detection and quantitation of biomarkers released from the brain, whether in serum or in phagocytes, is that they may be subject to a variety of alterations and modifications (alternate splice variants, post-translational modifications, or degradation) that may be specific for a particular disease state.

In order to eliminate these non-specific signals from the analysis of single cells, a more detailed analysis of the fluorescently stained PBMC cells used for the FM analysis was performed featuring a spectrally resolved fluorescence microscope equipped with an ultrashort-pulse laser enabling two-photon excitation that allows for excitation of the antibody labels (FITC and Texas Red) using near-infrared light. Because the emission spectrum for each of the fluorophores was in the visible range, using the two-photon microscope provided a significant separation (˜300-400 nm) between the excitation wavelength of the laser and the emission spectra of each of the fluorophores. This separation, which critically allows for acquisition of entire spectra of all the fluorescing species present, cannot be achieved with fluorescence microscopes employing single photon excitation. It is particularly advantageous when imaging cells exhibiting a significant level of autofluorescence (which has a rather broad spectrum), as it enables an exquisite discrimination between fluorescence from stained biomarkers and autofluorescence. The composite emission spectrum from each pixel in the set of spectrally resolved images was deconvoluted into green label for the >GFAP antibody, red label for the >CD14 antibody, and autofluorescence, using a previously published algorithm, along with the elementary spectra of each of the spectral components. The average autofluorescence spectrum was obtained by acquiring spectrally resolved fluorescence images of unstained cells. The deconvolution of pixel-level fluorescence generated separate spatial intensity maps of the green, red, and autofluorescence signals. The results of this analysis are shown in FIG. 4. Using the autofluorescence spatial intensity map, the outer boundary of each nucleated cell, identified by blue DAPI stain, was used to demarcate a region of interest (ROI). Then, in each ROI, clusters of pixels with similarly high intensity were identified in both the red and green intensity maps, using an automated algorithm, and the cluster of pixels with the highest average intensity within each map was chosen for each ROI. Finally, the pixel clusters were then organized according to mean red and green fluorescence intensity per pixel cluster. By raising the threshold for the GFAP-specific green fluorescence intensity to eliminate signals due to non-specific binding of the polyclonal antibody, 4 groups of cells become apparent (see FIG. 4); groups A and B, which are CD14 negative and groups C and D that are CD14 positive. The latter two groups (21% of all cells) appear to be monocytes (within the expected concentration for the composition of PBMC preparations), but only group D contains the GFAP biomarker. Of the CD14 negative cells, group A seems to represent regular lymphocytes, while group B might represent a mixture of non-classical monocytes, dendritic cells and neutrophils. Table 3 shows a summary with a comparison to the data in Table 2 above. While it could be argued that our threshold level, which was used to distinguish between specific and non-specific binding of the polyclonal >GFAP (green) antibody, is somewhat arbitrary, the pixel level analysis of the two-photon micro-spectrograms does appear to identify additional GFAP positive cells.

TABLE 3

| | PBMCs (DAPI+) | Fraction of PBMCs | Fraction of PBMCs | Fraction of CD14+ | Fraction of CD14− |
|---|---|---|---|---|---|
| | counted | that are CD14+ | that are GFAP+ | cells that are GFAP+ | cells that are GFAP+ |
| FM with cell | 15767 | 10.6% | 0.5% | 5.0% | 0.60% |
| area analysis | | | | | |

| | ROI's counted | Groups C and D | Groups B and D | Group D | Group B |
|---|---|---|---|---|---|
| FM with pixel | 1765 | 21% | 2.7% | 8.9% | 1.1% |
| level analysis | | | | | |

Also, FACS can be used to demonstrate that GFAP can be detected in peripheral blood phagocytes from a cognitively normal donor. After lysing red cells, WBCs were stained with a monoclonal antibody to GFAP as well as antibodies to cell surface markers CD14 and CD16, which allows the separation and classification of monocyte subgroups. Table 4 shows the results when gating out granulocytes and sorting cells by either their CD14 or CD16 surface markers and GFAP signal. A small fraction of GFAP positive cells (1.5%) was detected among the agranulocytes with various levels of CD14 and CD16 surface markers, suggesting that several different cell subgroups are involved in phagocytosis of this brain-specific biomarker.

TABLE 4

| | | | Relative | | | |
|---|---|---|---|---|---|---|
| | | | GFAP | % of gated | Intensity | |
| Gating | | # of cells | Intensity | cells | Ratio | Subgroup analysis |
| GFAP/CD14 | All | 44663 | 531 | 100% | | |
| | GFAP+ CD14+ | 131 | 1260 | 0.3% | 3.9 | all CD16− |
| | GFAP− CD14+ | 3307 | 325 | 7.4% | | mostly CD16− |
| | GFAP+ CD14− | 531 | 6140 | 1.2% | 13.0 | both CD16+ and CD16− |
| | GFAP− CD14− | 40289 | 474 | 90% | | mostly CD16+ |
| GFAP/CD16 | All | 44663 | 531 | 100% | | |
| | GFAP+ CD16+ | 230 | 5395 | 0.5% | 11.3 | all CD14− |
| | GFAP− CD16+ | 39089 | 476 | 88% | | all CD14− |
| | GFAP+CD16− | 438 | 4682 | 1.0% | 14.5 | mostly CD14− both also CD14+ |
| | GFAP− CD16− | 4681 | 324 | 10.5% | | mostly CD14+ |

As previously discussed, phagocytes containing brain biomarkers can be detected in blood from cognitively normal human donors. Single cell analysis can provide information on cell type and its biomarker load. Single cell analysis can be performed with whole blood without the need to isolate PBMCs. Polyclonal (or monoclonal) antibodies raised against specific epitopes on the biomarker molecule that are present in the phagocyte may be used to improve the accuracy of ELISA assays and avoid non-specific binding in single cell assays.

The present invention also includes optimized techniques for ELISA and single cell analysis. For example, the present invention includes the use of specific antibodies for biomarker (GFAP and Tau) epitopes in phagocytes.

Since phagocytes degrade their biomarker content over time, it is not expected that all native biomarker epitopes are present in a given cell or PBMC extract. Thus, the present invention includes a cocktail of epitope-specific antibodies against the epitopes, e.g., GFAP, Tau, etc. The epitopes may be, for example, epitopes that are most abundant in the phagocytes of cognitively normal donors.

Having multiple antibodies with mapped epitopes available may enable the development of sandwich ELISAs, where the primary antibody is immobilized on a surface (microplates, beads, slides, etc.). This assay format allows the user to add sample directly for capture of the biomarker(s), and is the preferred format for commercial assay kits. Moreover, this format may be helpful for assay automation, including the Isoplexis micro-chip based single cell proteomics assay discussed below.

Having particular GFAP and Tau antibodies in sufficiently large amounts may be helpful for high content screening/analysis with FM, FACS, or the more recent development of microplate-based systems (such as the Operetta CLS™ from PerkinElmer), which provide for additional speed and throughput. A novel and innovative tool for highly multiplexed single cell analysis is the microchip-based system from Isoplexis. Their Isocode chips combine single-cell proteomics of hundreds of cells in parallel with identification of cell subsets that secrete various proteins, which has been demonstrated to correlate with patient response to therapy.

As previously discussed, the present invention is not restricted to any given brain trauma or disease, but may rather be used to prove that measuring these biomarkers in people without any neurologic symptoms could become a standard addition to blood analysis. Being able to measure a ‘baseline’ of neurologic biomarker content in a person's blood easily and at low cost might allow the routine monitoring of brain health and early detection of changes that are prodromal for neurologic disease. In some embodiments, the concentration of biomarker per ml of blood may be correlated with the number of biomarker-containing phagocytes (e.g., broken down per cell subtype) in the same blood volume. Two-photon fluorescent microscopy may allow for the establishment of a range of biomarker load per cell. This may be helpful in studies of the effect of aging on biomarker status in phagocytes, since an age-related increase of glial biomarkers (independent of AD status), and particularly for Tau and p-Tau as a prodromal marker for AD, has been demonstrated in CSF and blood serum.

Aside from suitable antibodies, sample preparation is important. The proteomic analysis of white blood cells (WBCs), and especially that of small cell subsets, may require some enrichment, if not the isolation of those cells via gradient centrifugation or cell sorting. The present invention has described ficoll gradients to separate PBMCs from granulocytes and red cells for ELISA assays. In some embodiments, having identified the specific cell types which contain brain biomarkers through single cell analysis, the cell enrichment may be simplified using their surface antigens for capture on functionalized magnetic beads for ELISA, on coated microplate surfaces for HCS, and on coated slides for FM, etc. For ELISA, this task is straightforward since magnetic beads functionalized with various cell surface markers are widely available for both positive and negative selection. The present invention includes functionalizing microplate wells and microscope slides for rapid capture of phagocytes. For example, the methods and systems herein may be able to capture sufficient cells for brain biomarker analysis from a few drops of blood, e.g., finger prick collection instead of venipuncture. There are about 15,000 monocytes per drop (0.1 ml) of blood, and assuming that 0.1% of those contain biomarkers, as our results suggest, it may be possible to capture a sufficient number of cells from a few drops of blood for single cell analysis. Fixing/permeabilization and immunostaining of captured cells could be performed manually with a few steps and in relatively short time and provide a path for future adoption by automated systems for slide staining or microplate handling.

### EXAMPLE 7

The following describes methods, compositions, and applications of the present invention. The present invention is not limited to the methods or materials described herein.

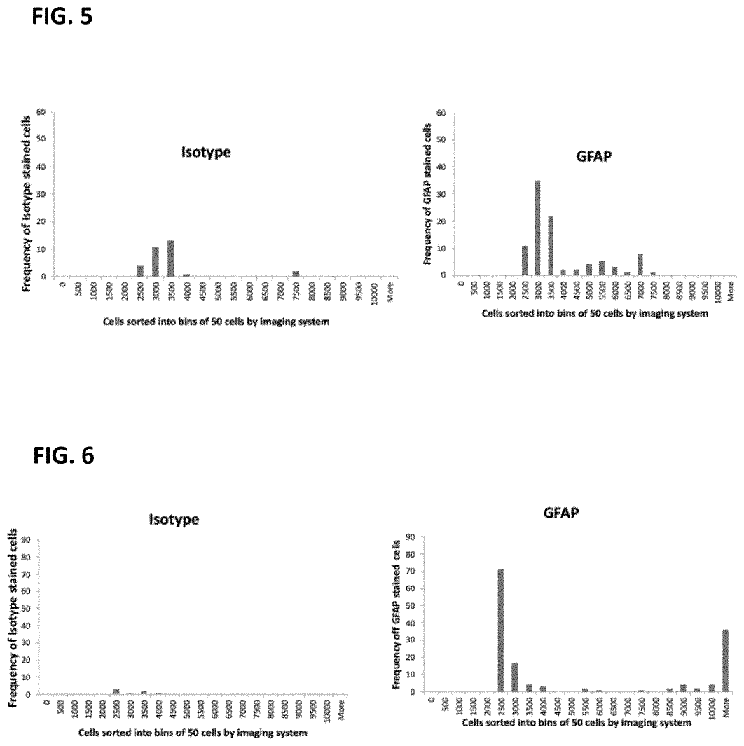

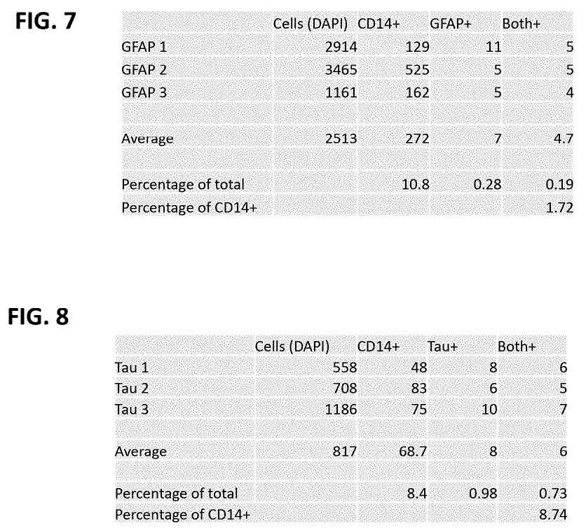

FIG. 5 and FIG. 6 show an example of single cell analysis by imaging of fluorescently labeled cells. Results shown are from a collaborative experiment where rats were used to test the effect of brain surgery followed by implantation of electrodes. Control rats received only the brain surgery (opening the skull without affecting the brain tissue). PBMC preparations from two rats each were pooled and analyzed on the Operetta (Operetta CLS High-Content Analysis System from Perkin Elmer). The pooled blood samples were treated with propidium iodide to stain nuclei (orange), followed by differentially staining PBMC cells with fluorescently labeled antibodies to monocyte-specific cell surface markers CD43 (red) and CD11 b/c (blue), whereby the presence or absence of these surface proteins identify specific cell subtypes.

To test for the presence of the brain-specific biomarker GFAP, cells were also reacted first with a primary antibody specific for GFAP (rabbit polyclonal anti-GFAP from Encor Biotechnology), followed by a fluorescently labeled goat anti-rabbit antibody (green; from Thermofisher) for detection. Alternatively, a rabbit Isotype antibody (rabbit IgG from Biolegend) was used as a control for non-specific binding. This antibody was also detected by reaction with the fluorescently labeled Goat anti-Rabbit antibody.

FIG. 5 shows the results from the rats that received brain surgery but no electrode implants. Results are shown for cells that were CD43 negative and CD11b/c positive (subset of monocytes) and positive for either GFAP or Isotype. (0.67% of all PBMC cells analyzed were found to stain with the Isotype; 0.76% of all PBMC cells analyzed were found to stain with the anti-GFAP antibody.) No significant difference was found between Isotype and GFAP stained PBMC preps, suggesting that the surgery itself does not lead to brain inflammation.

FIG. 6 shows the results from the rats that received the brain surgery including the electrode implants. Results are shown for cells that were CD43 negative and CD11b/c positive (subset of monocytes) and positive for either GFAP or Isotype. (0.17% of all PBMC cells analyzed were found to stain with the Isotype; 1.82% of all PBMC cells analyzed were found to stain with the anti-GFAP antibody.) The number of cells staining positive for the brain-specific biomarker GFAP is 10 fold higher than for the Isotype stained cells, suggesting that the implantation of electrodes caused brain inflammation.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method, comprising:
a. producing a preparation comprising CNS-derived molecules by introducing to a whole blood sample obtained from outside central nervous system (CNS) tissue of a subject a first detectable binding moiety specific for circulating phagocytes and a second detectable binding moiety specific for a CNS-derived molecule, the first detectable binding moiety being differentially detectable from the second detectable binding moiety;
b. subjecting the preparation to single-cell analysis for detecting the first detectable binding moiety and second detectable binding moiety; and
c. analyzing the CNS-derived molecules in the preparation.

2. The method of claim 1, wherein the single-cell analysis is flow cytometry.

3. The method of claim 1, wherein the single-cell analysis is a single cell enzyme linked immunosorbent assay (ELISA).

4. The method of claim 1, wherein the single-cell analysis is a microscopy-based assay.

5. The method of claim 1, wherein single-cell analysis comprises placing the preparation on a solid surface, using said surface as a wave guide for illumination, and imaging by direct charge-coupled device (CCD).

6. The method of claim 1, wherein the CNS-derived compounds are peptides, whole proteins, epitopes of a protein or peptide, lipids, membrane components, nucleic acids, metabolites, toxins, infectious agents, or a combination thereof.

7. The method of claim 1, wherein the circulating phagocytes are macrophages, monocytes or a subgroup thereof, dendritic cells, neutrophils, or a combination thereof.

8. The method of claim 1, wherein the first detectable binding moiety, the second detectable binding moiety, or both comprise a fluorescent label, a fluorescent antibody, a nanoparticle, a quantum dot, or a tag.

9. The method of claim 1, wherein the CNS-derived molecule comprises one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin All, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.

10. A method, comprising:
a. producing a preparation comprising CNS-derived molecules by introducing to a whole blood sample obtained from outside central nervous system (CNS) tissue of a subject a first detectable binding moiety specific for circulating phagocytes and a second detectable binding moiety specific for a CNS-derived molecule, the first detectable binding moiety being differentially detectable from the second detectable binding moiety; and
b. subjecting the preparation to single-cell analysis for detecting the first detectable binding moiety and second detectable binding moiety;
c. analyzing the CNS-derived molecules in the preparation using flow cytometry, microscopy, or direct charge-coupled device (CCD).

11. The method of claim 10, wherein the single-cell analysis is flow cytometry, a single cell enzyme linked immunosorbent assay (ELISA), or a microscopy-based assay.

12. The method of claim 10, wherein the CNS-derived compounds are peptides, whole proteins, epitopes of a protein or peptide, lipids, membrane components, nucleic acids, metabolites, toxins, infectious agents, or a combination thereof.

13. The method of claim 10, wherein the circulating phagocytes are macrophages, monocytes or a subgroup thereof, dendritic cells, neutrophils, or a combination thereof.

14. The method of claim 10, wherein the first detectable binding moiety, the second detectable binding moiety, or both comprise a fluorescent label, a fluorescent antibody, a nanoparticle, a quantum dot, or a tag.

15. The method of claim 10, wherein the CNS-derived molecule is Tau or GFAP.

16. The method of claim 10, wherein the CNS-derived molecule comprises one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin All, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.

17. A method, comprising:
a. producing a preparation comprising CNS-derived molecules by introducing to a whole blood sample obtained from outside central nervous system (CNS) tissue of a subject a first detectable binding moiety specific for circulating phagocytes and a second detectable binding moiety specific for a CNS-derived molecule, the first detectable binding moiety being differentially detectable from the second detectable binding moiety; and
b. analyzing the CNS-derived molecules in the preparation.

18. The method of claim 17, wherein the preparation is produced for single-cell analysis of the CNS-derived molecules.

19. The method of claim 17, wherein the CNS-derived molecule is Tau, GFAP, or a combination thereof.

20. The method of claim 17, wherein the CNS-derived molecule comprises one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin All, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.
